(19) United States
(12) Patent Application Publication
(10) Pub. No.: US 2022/0265654 A1
(43) Pub. Date: Aug. 25, 2022

(54) DOSAGE REGIMENS FOR PARP7 INHIBITORS

(71) Applicant: Ribon Therapeutics, Inc., Cambridge, MA (US)

(72) Inventors: Melissa Marie Vasbinder, Newton, MA (US); Lucas J. Utley, Boston, MA (US); Viviana Bozon, West Newton, MA (US); Kevin Wayne Kuntz, Woburn, MA (US); Sudha Parasuraman, Lexington, MA (US); William McCulloch, Raleigh, NC (US); Nolan Wood, Sandwich (GB)

(21) Appl. No.: 17/671,714

(22) Filed: Feb. 15, 2022

Related U.S. Application Data
(60) Provisional application No. 63/149,901, filed on Feb. 16, 2021.
(60) Provisional application No. 63/275,163, filed on Nov. 3, 2021.

(51) Int. Cl. A61K 31/506; A61K 45/06; A61K 9/14

(52) U.S. Cl. CPC A61K 31/506; A61K 9/14; A61K 45/06

(57) ABSTRACT

The present invention is directed to dosing, dosage regimens, formulations, unit dosage forms, and related, of a PARP7 inhibitor for the treatment of cancer. The present invention is also directed to methods of treating cancer by administering the PARP7 inhibitor in combination with an antibody that binds to PD-1.

### CLAIM OF PRIORITY

This application claims priority to U.S. Patent Application Ser. No. 63/149,901, filed on Feb. 16, 2021 and U.S. Patent Application Ser. No. 63/275,163, filed on Nov. 3, 2021. The entire contents of the applications are hereby incorporated by reference.

### FIELD OF THE INVENTION

The present invention is directed to dosing, dosage regimens, formulations, unit dosage forms, and related, of a PARP7 inhibitor for the treatment of cancer. The present invention is also directed to methods of treating cancer by administering the PARP7 inhibitor in combination with an antibody that binds to PD-1.

### BACKGROUND OF THE INVENTION

Poly(ADP-ribose) polymerases (PARPs) are members of a family of seventeen enzymes that regulate fundamental cellular processes including gene expression, protein degradation, and multiple cellular stress responses (M. S. Cohen, P. Chang, Insights into the biogenesis, function, and regulation of ADP-ribosylation. Nat Chem Biol 14, 236-243 (2018)). PARP7 is a member of the monoPARP family of proteins, which are key regulators of stress responses. In cancer cells, genetic instability causes leakage of nucleic acid in the cytoplasm. PARP7 over expression, which is characteristic of many cancer types, leads to suppressed nucleic acid sensing. This enables cancer cells to keep proliferating and evade the immune system.

PARP7 inhibitors have been described as useful in the treatment of cancer. See, for example, U.S. Pat. No. 10,550,105. Given the role PARP7 over expression has in cancer development and progression, it is evident that a suitable and effective dosage regimen for administering a PARP7 inhibitor to a patient for the treatment of cancer is necessary. The dosing regimens, unit dosage forms, and related embodiments described herein help address this need.

### SUMMARY OF THE INVENTION

The present invention relates to a method for treating cancer in a subject, wherein the method comprises administering to the subject the compound 5-[[(2S)-1-(3-oxo-3-[4-[5-(trifluoromethyl)pyrimidin-2-yl]piperazin-1-yl]propoxy)propan-2-yl]amino]-4-(trifluoromethyl)-2,3-dihydropyridazin-3-one (the compound of Formula I), or a pharmaceutically acceptable salt thereof, at a specified total daily dosage.

The present invention further provides a unit dosage form comprising the compound of Formula I, or a pharmaceutically acceptable salt thereof, in specified amounts.

The present invention further provides a method for treating cancer in a human subject, wherein the method comprises administering to the subject a pharmaceutical composition that comprises the compound of Formula I, or a pharmaceutically acceptable salt thereof, in an amount sufficient to provide at steady-state a mean Cₘₐₓ, a mean Cₘᵢₙ, a mean Tₘₐₓ, or a mean AUC_{0-12 h} at specified values.

The invention further provides a solid preparation of the compound of Formula I, or a pharmaceutically acceptable salt thereof, which is micronized.

The invention further provides a solid pharmaceutical formulation comprising the compound of Formula I, or a pharmaceutically acceptable salt thereof, in a specified amount, and at least one pharmaceutically acceptable excipient.

The invention further provides a method for treating cancer in a subject, wherein the method comprises administering to the subject:

(i) the compound 5-[[(2S)-1-(3-oxo-3-[4-[5-(trifluoromethyl)pyrimidin-2-yl]piperazin-1-yl]propoxy)propan-2-yl]amino]-4-(trifluoromethyl)-2,3-dihydropyridazin-3-one, or a pharmaceutically acceptable salt thereof; and

(ii) an antibody that binds to human PD-1.

### BRIEF DESCRIPTION OF DRAWINGS

FIG. 1A is a graph showing mean plasma profiles for doses of 25 mg-500 mg BID in humans on Day 1 of administration of the compound of Formula I.

FIG. 1B is a graph showing mean plasma profiles for doses of 25 mg-500 mg BID in humans on Day 15 of administration of the compound of Formula I.

FIG. 2 is a table showing data from FIGS. 1A-1B.

FIG. 3 is a graph showing mean plasma profiles for dogs treated with 100 mg of micronized and nonmicronized forms of the compound of Formula I.

### DETAILED DESCRIPTION

### Dosage Regimens

The present invention is directed to, inter alia, dosage regimens for the treatment of cancer by administration of a PARP7 inhibitor of Formula I.

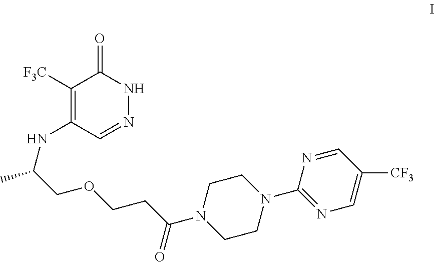 [SMILES: C[C@@H](COCCC(=O)N1CCN(c2ncc(C(F)(F)F)cn2)CC1)Nc1cn[nH]c(=O)c1C(F)(F)F]

or a pharmaceutically acceptable salt thereof. The chemical name for the compound of Formula I is 5-[[(2S)-1-(3-oxo-3-[4-[5-(trifluoromethyl)pyrimidin-2-yl]piperazin-1-yl]propoxy)propan-2-yl]amino]-4-(trifluoromethyl)-2,3-dihydropyridazin-3-one. This compound, and its pharmaceutically salts, are described in U.S. Pat. No. 10,550,105, the disclosure of which is incorporated by reference in its entirety.

The compound of Formula I, or a pharmaceutically acceptable salt thereof, can be administered according to a dosage regimen described herein to treat a PARP7-associated disorder like, for example, cancer in a subject. Accordingly, the present invention provides a method for treating cancer in a subject, wherein the method includes administering to the subject a compound of Formula I, or a pharmaceutically acceptable salt thereof, at a total daily dosage of about 50 mg to about 1000 mg, measured as the free base. In some embodiments, the compound, or a pharmaceutically acceptable salt thereof, is administered at a total daily dosage of about 50 mg, about 100 mg, about 200 mg, about 400 mg, about 600 mg, about 800 mg, or about 1000 mg measured as the free base. In some embodiments, the compound, or a pharmaceutically acceptable salt thereof, is administered at a total daily dosage of about 50 mg, about 100 mg, about 200 mg, about 400 mg, about 600 mg, about 800 mg, or about 1000 mg. In some embodiments, the compound, or a pharmaceutically acceptable salt thereof, is administered at a total daily dosage of about 50 mg measured as the free base. In some embodiments, the compound, or a pharmaceutically acceptable salt thereof, is administered at a total daily dosage of about 100 mg measured as the free base. In some embodiments, the compound, or a pharmaceutically acceptable salt thereof, is administered at a total daily dosage of about 200 mg measured as the free base. In some embodiments, the compound, or a pharmaceutically acceptable salt thereof, is administered at a total daily dosage of about 300 mg measured as the free base. In some embodiments, the compound, or a pharmaceutically acceptable salt thereof, is administered at a total daily dosage of about 400 mg measured as the free base. In some embodiments, the compound, or a pharmaceutically acceptable salt thereof, is administered at a total daily dosage of about 500 mg measured as the free base. In some embodiments, the compound, or a pharmaceutically acceptable salt thereof, is administered at a total daily dosage of about 600 mg measured as the free base. In some embodiments, the compound, or a pharmaceutically acceptable salt thereof, is administered at a total daily dosage of about 700 mg measured as the free base. In some embodiments, the compound, or a pharmaceutically acceptable salt thereof, is administered at a total daily dosage of about 800 mg measured as the free base. In some embodiments, the compound, or a pharmaceutically acceptable salt thereof, is administered at a total daily dosage of about 900 mg measured as the free base. In some embodiments, the compound, or a pharmaceutically acceptable salt thereof, is administered at a total daily dosage of about 1000 mg measured as the free base.

In some instances, the compound of Formula I, or a pharmaceutically acceptable salt thereof, is administered once daily. In other instances, the compound of Formula I, or pharmaceutically acceptable salt thereof, is administered twice daily.

In some examples, the compound of Formula I, or a pharmaceutically acceptable salt thereof, is administered according to a continuous dosing schedule. For instance, the continuous dosing schedule includes administering the compound, or a pharmaceutically acceptable salt thereof, to the subject for two or more consecutive days. In some embodiments, the continuous dosing schedule includes administering the compound, or a pharmaceutically acceptable salt thereof, to the subject for five or more consecutive days. In other embodiments, the continuous dosing schedule includes administering the compound, or a pharmaceutically acceptable salt thereof, to the subject for ten or more consecutive days. In some embodiments, the continuous dosing schedule includes administering the compound, or a pharmaceutically acceptable salt thereof, to the subject for fifteen or more consecutive days. In some examples, the continuous dosing schedule includes administering the compound, or a pharmaceutically acceptable salt thereof, to the subject for twenty or more consecutive days. In some embodiments, the continuous dosing schedule includes administering the compound, or a pharmaceutically acceptable salt thereof, to the subject for twenty-one or more consecutive days.

In some embodiments, the compound of Formula I, or a pharmaceutically acceptable salt thereof, is administered according to an intermittent dosing schedule. In some examples, the intermittent dosing schedule includes administering the compound, or a pharmaceutically acceptable salt thereof, to the subject for a treatment period of two or more consecutive days followed by a period of at least one day of no treatment with the compound, or a pharmaceutically acceptable salt thereof. In some instances, the treatment period is at least seven consecutive days. In some examples, the treatment period is 7 to 21 consecutive days. In some instances, the treatment period is 14 consecutive days. In some embodiments, the period of no treatment is at least 2 consecutive days. In some instances, the period of no treatment is at least 4 consecutive days. In other instances, the period of no treatment is 4 to 10 consecutive days. In some embodiments, the period of no treatment is 7 consecutive days. In some embodiments, the intermittent dosing schedule includes at least two treatment periods, where all treatment periods are separated by a period of no treatment.

In some embodiments, the patient is administered about 25 mg, about 50 mg, about 100 mg, about 200 mg, about 300 mg, about 400 mg, or about 500 mg of the compound of Formula I, or a pharmaceutically acceptable salt thereof, measured as the free base, twice daily. In some embodiments, the patient is administered about 200 mg or about 300 mg of the compound of Formula I, or a pharmaceutically acceptable salt thereof, measured as the free base, twice daily. In some embodiments, the patient is administered about 200 mg of the compound of Formula I, or a pharmaceutically acceptable salt thereof, measured as the free base, twice daily. In some embodiments, the patient is administered about 300 mg of the compound of Formula I, or a pharmaceutically acceptable salt thereof, measured as the free base, twice daily. In some embodiments, the patient is administered about 100 mg of the compound of Formula I, or a pharmaceutically acceptable salt thereof, measured as the free base, twice daily.

In some embodiments, the patient is administered about 25 mg, about 50 mg, about 100 mg, about 200 mg, about 300 mg, about 400 mg, or about 500 mg of the compound of Formula I, or a pharmaceutically acceptable salt thereof, measured as the free base, once daily. In some embodiments, the patient is administered about 200 mg or about 300 mg of the compound of Formula I, or a pharmaceutically acceptable salt thereof, measured as the free base, once daily. In some embodiments, the patient is administered about 100 mg of the compound of Formula I, or a pharmaceutically acceptable salt thereof, measured as the free base, once daily. In some embodiments, the patient is administered about 200 mg of the compound of Formula I, or a pharmaceutically acceptable salt thereof, measured as the free base, once daily.

### Pharmaceutical Unit Dosage Forms, Formulations, and Kits

Also provided herein is a unit dosage form containing the compound of Formula I, or a pharmaceutically acceptable salt thereof, in an amount of about 25 mg to about 500 mg, about 100 mg to about 400 mg, or about 200 mg to about 300 mg, measured as the free base. In some instances, the unit dosage form includes the compound of Formula I, or a pharmaceutically acceptable salt thereof, in an amount of about 25 mg, about 50 mg, about 75 mg, about 100 mg, about 150 mg, about 200 mg, about 250 mg, about 300 mg, about 400 mg, or about 500 mg, measured as the free base. In some instances, the unit dosage form, is suitable for oral administration, such as in the form of a tablet or capsule.

Formulations for the preparation of unit dosage forms and for pharmaceutical administration of the compound of Formula I, or a pharmaceutically acceptable salt thereof, can comprise about 30-50%, about 35 to 45%, or about 40% w/w of the compound of Formula I, or a pharmaceutically acceptable salt thereof.

The term “unit dosage forms” refers to physically discrete units suitable as unitary dosages for human patients and other mammals, each unit containing a predetermined quantity of active material calculated to produce the desired therapeutic effect, in association with a suitable pharmaceutically acceptable carrier.

As used herein, the term “pharmaceutically acceptable salt” is intended to apply to any salt, whether previously known or future discovered, that is used by one skilled in the art that is a non-toxic organic or inorganic addition salt, which is suitable for use as a pharmaceutical. “Pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

In some embodiments, the compound of Formula I, or a pharmaceutically acceptable salt thereof, is formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for oral, intravenous, intramuscular, subcutaneous, parenteral, rectal, spinal or epidermal administration (e.g. by injection or infusion). The exact proportion of carriers/excipients, as described herein, is determined by the solubility and chemical properties of the active compound, the chosen route of administration as well as standard pharmaceutical practice.

For preparing solid compositions such as tablets, the principal active ingredient is mixed with a pharmaceutically acceptable excipient or carrier to form a solid pre-formulation composition containing a homogeneous mixture of a compound of the present disclosure. As used herein, the phrase “pharmaceutically acceptable excipient” refers to a pharmaceutically-acceptable material, composition, or vehicle, such as a liquid or solid filler, diluent, solvent, or encapsulating material. Excipients are generally safe, non-toxic and neither biologically nor otherwise undesirable and include excipients that are acceptable for veterinary use as well as human pharmaceutical use. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above.

Also within the scope of the disclosure is a kit including the compound of Formula I or a pharmaceutically acceptable salt thereof, a composition, or unit dosage form described herein. The kit can include one or more other elements including: instructions for use (e.g., in accordance a dosage regimen described herein); other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

### Particle Size and Micronization

The compound of Formula I can be prepared in micronized and nonmicronized forms. In some embodiments, the compound of Formula I used in the dosage forms of the invention is micronized. The micronized drug substance can be prepared, for example, by jet milling. Particle size can be measured by laser diffraction methods (e.g., USP 42 <429> Light Diffraction Measurement of Particle Size). Nonmicronized compound can have a particle diameter size [d90] of about 30 μm or larger. In some embodiments, nonmicronized compound has a particle diameter size [d90] of about 30 to about 500 μm, about 50 to about 400 μm, about 100 to about 350 μm, or about 150 to about 300 μm. Micronized compound can have a particle diameter size [d90] less than about 30 μm. In some embodiments, micronized compound can have a particle diameter size [d90] of about 1 to about 20 μm, about 2 to about 18 μm, about 5 to about 15 μm, or about 7 to about 12 μm. In some embodiments, micronized compound can have a particle diameter size [d90] of about 8, 9, 10, or 11 μm.

In some embodiments, the compound of Formula I used in the dosage forms and formulations of the invention is not micronized, but is prepared in a manner that results in a small particle size, such as having a particle diameter size [d90] less than about 30 μm. In some embodiments, nonmicronized compound can have a particle diameter size [d90] of about 1 to about 20 μm, about 2 to about 18 μm, about 5 to about 15 μm, or about 7 to about 12 μm. In some embodiments, nonmicronized compound can have a particle diameter size [d90] of about 8, 9, 10, or 11 μm.

In some embodiments, the compound of Formula I, or a pharmaceutically acceptable salt thereof, having a small particle size (via micronization or not) is used to prepare the pharmaceutical formulations and solid dosage forms described herein. In some embodiments, patients are administered the compound of Formula I, or a pharmaceutically acceptable salt thereof, having a small particle size. In some embodiments, patients are administered about 100 mg to about 300 mg, about 150 to about 250 mg, or about 200 mg of the compound of Formula I, or a pharmaceutically acceptable salt thereof, having a particle diameter [d90] of less than about 30 μm, measured as the free base, twice daily. In some embodiments, administered compound, or a pharmaceutically acceptable salt thereof, can have a particle diameter size [d90] of about 1 to about 20 μm, about 2 to about 18 μm, about 5 to about 15 μm, or about 7 to about 12 μm. In some embodiments, administered compound, or a pharmaceutically acceptable salt thereof, can have a particle diameter size [d90] of about 8, 9, 10, or 11 μm. The administered compound, or pharmaceutically acceptable salt thereof, can have been micronized to achieve the small particle size.

The present invention further provides a process of reducing the particle size of a preparation of the compound of Formula I, or a pharmaceutically acceptable salt thereof, comprising micronizing the nonmicronized preparation of the compound of Formula I, or a pharmaceutically acceptable salt thereof. In some embodiments, the nonmicronized preparation of the compound of Formula I, or a pharmaceutically acceptable salt thereof has a particle diameter [d90] of about 30 μm or more. In some embodiments, the nonmicronized preparation of the compound of Formula I, or a pharmaceutically acceptable salt thereof has a particle diameter [d90] of about 30 μm to about 500 μm, about 50 μm to about 400 μm, about 100 μm to about 350 μm, or about 150 μm to about 300 μm. In some embodiments, the resulting micronized compound, or a pharmaceutically acceptable salt thereof, can have a particle diameter size [d90] of less than about 30 μm, for example, about 1 to about 20 μm, about 2 to about 18 μm, about 5 to about 15 μm, or about 7 to about 12 μm. In some embodiments, the resulting micronized compound, or a pharmaceutically acceptable salt thereof, can have a particle diameter size [d90] of about 8, 9, 10, or 11 μm. The present invention further provides the micronized product prepared by any of the processes described above.

The present invention further provides a method of increasing bioavailability of the compound of Formula I, or a pharmaceutically acceptable salt thereof, comprising micronizing the compound of Formula I, or a pharmaceutically acceptable salt thereof, prior to administration to a subject, where the increasing bioavailability is relative to the bioavailability of the same amount of nonmicronized compound of Formula I, or a pharmaceutically acceptable salt thereof. The increased bioavailability can be measured by an increase in mean Cₘₐₓ or mean AUC. For example, mean Cₘₐₓ can be increased by about 1.5 to about 3-fold, about 2 to about 3-fold, or about 2.4-fold. Additionally, mean AUC can be increased by about 1.2 to about 2-fold, about 1.3 to about 1.8-fold, or about 1.5 fold.

### Pharmacokinetics

Also provided herein, are methods for treating cancer in a subject, wherein the method includes administering to the subject a pharmaceutical composition that includes the compound of Formula I, or a pharmaceutically acceptable salt thereof, in micronized or nonmicronized form, an amount sufficient to provide at steady-state:

(a) a mean Cₘₐₓ of about 140 ng/mL to about 1600 ng/mL;

(b) a mean Cₘᵢₙ of at least about 50 ng/mL to 700 ng/mL;

(c) a mean Tₘₐₓ of about 1 hour to 5 hours; or

(d) a mean AUC_{0-12 h} of about 800 hr*ng/mL to 10500 hr*ng/mL.

In some embodiments, the method includes administering to the subject a pharmaceutical composition that includes the compound of Formula I, or a pharmaceutically acceptable salt thereof, in micronized or nonmicronized form, an amount sufficient to provide at steady-state:

(a) a mean Cₘₐₓ of about 200 ng/mL to about 800 ng/mL;

(b) a mean Cₘᵢₙ of at least about 200 ng/mL to 500 ng/mL;

(c) a mean Tₘₐₓ of about 2 hours to 4 hours; or

(d) a mean AUC_{0-12 h} of about 1000 hr*ng/mL to 5000 hr*ng/mL.

In some embodiments, the method includes administering to the subject a pharmaceutical composition that includes the compound of Formula I, or a pharmaceutically acceptable salt thereof, in micronized or nonmicronized form, an amount sufficient to provide at steady-state:

(a) a mean Cₘₐₓ of about 400 ng/mL to about 700 ng/mL;

(b) a mean Cₘᵢₙ of at least about 300 ng/mL to 500 ng/mL;

(c) a mean Tₘₐₓ of about 2 hours to 3 hours; or

(d) a mean AUC_{0-12 h} of about 2000 hr*ng/mL to 5000 hr*ng/mL.

In some embodiments, the method includes administering to the subject a pharmaceutical composition that includes the compound of Formula I, or a pharmaceutically acceptable salt thereof, in micronized or nonmicronized form, an amount sufficient to provide at steady-state:

(a) a mean Cₘₐₓ of about 500 ng/mL to about 700 ng/mL;

(b) a mean Cₘᵢₙ of at least about 300 ng/mL to 500 ng/mL;

(c) a mean Tₘₐₓ of about 2 hours to 3 hours; or

(d) a mean AUC_{0-12 h} of about 3000 hr*ng/mL to 4200 hr*ng/mL.

In some embodiments, the method includes administering to the subject a pharmaceutical composition that includes the compound of Formula I, or a pharmaceutically acceptable salt thereof, in micronized or nonmicronized form, an amount sufficient to provide at steady-state:

(a) a mean Cₘₐₓ of about 500 ng/mL to about 700 ng/mL, or

(d) a mean AUC_{0-12 h} of about 3000 hr*ng/mL to 4200 hr*ng/mL.

In some embodiments, the compound of Formula I, or a pharmaceutically acceptable salt thereof, in micronized or nonmicronized form, is administered in an amount sufficient to provide at steady-state, a mean Cₘₐₓ of about 200 ng/mL to about 800 ng/mL. In some embodiments, the compound of Formula I, or a pharmaceutically acceptable salt thereof, in micronized or nonmicronized form, is administered in an amount sufficient to provide at steady-state, a mean Cₘₐₓ of about 300 ng/mL to about 800 ng/mL. In some embodiments, the compound of Formula I, or a pharmaceutically acceptable salt thereof, in micronized or nonmicronized form, is administered in an amount sufficient to provide at steady-state, a mean Cₘₐₓ of about 400 ng/mL to about 700 ng/mL. In some embodiments, the compound of Formula I, or a pharmaceutically acceptable salt thereof, in micronized or nonmicronized form, is administered in an amount sufficient to provide at steady-state, a mean Cₘₐₓ of about 500 ng/mL to about 700 ng/mL. In some embodiments, compound of Formula I, or a pharmaceutically acceptable salt thereof, in micronized or nonmicronized form, is administered in an amount sufficient to provide at steady-state, a mean Cₘₐₓ of about 600 ng/mL to about 700 ng/mL.

In some embodiments, the compound of Formula I, or a pharmaceutically acceptable salt thereof, in micronized or nonmicronized form, is administered in an amount sufficient to provide at steady-state, a mean Cₘᵢₙ of about 200 ng/mL to about 500 ng/mL. In some embodiments, the compound of Formula I, or a pharmaceutically acceptable salt thereof, in micronized or nonmicronized form, is administered in an amount sufficient to provide at steady-state, a mean Cₘᵢₙ of about 300 ng/mL to about 500 ng/mL. In some embodiments, the compound of Formula I, or a pharmaceutically acceptable salt thereof, in micronized or nonmicronized form, is administered in an amount sufficient to provide at steady-state, a mean Cₘᵢₙ of about 300 ng/mL to about 400 ng/mL.

In some embodiments, the compound of Formula I, or a pharmaceutically acceptable salt thereof, in micronized or nonmicronized form, is administered in an amount sufficient to provide at steady-state, a mean Tₘₐₓ of about 2 hours to 4 hours. In some embodiments, the compound of Formula I, or a pharmaceutically acceptable salt thereof, in micronized or nonmicronized form, is administered in an amount sufficient to provide at steady-state, a mean Tₘₐₓ of about 2 hours to 3 hours.

In some embodiments, the compound of Formula I, or a pharmaceutically acceptable salt thereof, in micronized or nonmicronized form, is administered in an amount sufficient to provide at steady-state, a mean AUC_{0-12 h} of about 1000 hr*ng/mL to 5000 hr*ng/mL. In some embodiments, the compound of Formula I, or a pharmaceutically acceptable salt thereof, in micronized or nonmicronized form, is administered in an amount sufficient to provide at steady-state, a mean AUC_{0-12 h} of about 2000 hr*ng/mL to 5000 hr*ng/mL. In some embodiments, the compound of Formula I, or a pharmaceutically acceptable salt thereof, in micronized or nonmicronized form, is administered in an amount sufficient to provide at steady-state, a mean AUC_{0-12 h} of about 2500 hr*ng/mL to 4500 hr*ng/mL. In some embodiments, the compound of Formula I, or a pharmaceutically acceptable salt thereof, in micronized or nonmicronized form, is administered in an amount sufficient to provide at steady-state, a mean AUC_{0-12 h} of about 3000 hr*ng/mL to 4200 hr*ng/mL. In some embodiments, the compound of Formula I, or a pharmaceutically acceptable salt thereof, in micronized or nonmicronized form, is administered in an amount sufficient to provide at steady-state, a mean AUC_{0-12 h} of about 1400 hr*ng/mL to 4500 hr*ng/mL. In some embodiments, the compound of Formula I, or a pharmaceutically acceptable salt thereof, in micronized or nonmicronized form, is administered in an amount sufficient to provide at steady-state, a mean AUC_{0-12 h} of about 1200 hr*ng/mL to 4000 hr*ng/mL.

### Methods of Treatment

Provided herein are methods for the treatment of cancer in a subject in need thereof by administering to the subject the compound of Formula I, or a pharmaceutically acceptable salt thereof, in micronized or nonmicronized form, in a unit dosage form, as disclosed herein, and/or in accordance with a dosage regimen disclosed herein, such as by a continuous dosing schedule or an intermittent dosing schedule. As described herein, a continuous dosing schedule refers to where the subject is administered a dosage continuously, e.g., substantially uninterrupted, for the entire treatment, for example, for two or more consecutive days, or for five or more consecutive days, or for ten or more consecutive days, or for fifteen or more consecutive days, or for twenty or more consecutive days, or for twenty-one or more days consecutively, or longer. A period of treatment on a continuous dosing schedule can be anywhere from days, weeks, or months. As described herein, an intermittent dosing schedule refers to where the subject is administered a dosage during a period of time which is interrupted by one or more periods of time of no treatment with the compound. Stated differently, intermittent dosing schedules refer to those in which the subject is administering the compound of Formula I, or a pharmaceutically acceptable salt thereof, to the subject for a treatment period of two or more consecutive days followed by a period of at least one day of no treatment with the compound, or a pharmaceutically acceptable salt thereof. The treatment period may be anywhere from 7 consecutive days to 21 consecutive days, for example, at least two consecutive days, at least seven consecutive days, or at least fourteen (14) consecutive days. The period of no treatment may be anywhere from 4 to 10 consecutive days, for example, at least 2 consecutive days, at least 4 consecutive days, at least 7 consecutive days.

As used herein, the terms “treat,” “treatment,” and “treating” refer to the reduction or amelioration of the progression, severity and/or duration of a disorder, e.g., a proliferative disorder, or the amelioration of one or more symptoms (preferably, one or more discernible symptoms) of the disorder resulting from the administration of one or more therapies. In specific embodiments, the terms “treat,” “treatment,” and “treating” refer to the amelioration of at least one measurable physical parameter of a proliferative disorder, such as growth of a tumor, not necessarily discernible by the patient. In other embodiments the terms “treat,” “treatment,” and “treating” refer to the inhibition of the progression of a proliferative disorder, either physically by, e.g., stabilization of a discernible symptom, physiologically by, e.g., stabilization of a physical parameter, or both. In other embodiments the terms “treat,” “treatment,” and “treating” refer to the reduction or stabilization of tumor size or cancerous cell count.

As described herein, the terms ‘subject’ or ‘patient’ refers to a warm blooded animal such as a mammal which is afflicted with a particular disease, disorder or condition. It is explicitly understood that guinea pigs, dogs, cats, rats, mice, horses, cattle, sheep, and humans are examples of animals within the scope of the meaning of the term.

The term “cancer” refers to a disease characterized by the rapid and uncontrolled growth of aberrant cells. Cancer cells can spread locally or through the bloodstream and lymphatic system to other parts of the body. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors. As used herein, the term “cancer” is meant to include all types of cancerous growths or oncogenic processes, metastatic tissues or malignantly transformed cells, tissues, or organs, irrespective of histopathological type or stage of invasiveness. Examples of cancerous disorders include, but are not limited to, breast, central nervous system, endometrium, kidney, large intestine, lung, oesophagus, ovary, pancreas, prostate, stomach, head and neck (upper aerodigestive), urinary tract, colon, cancers in which PARP7 expression is amplified, and others. In some embodiments, the cancers treatable according to the present invention include hematopoietic malignancies such as leukemia and lymphoma. Example lymphomas include Hodgkin's or non-Hodgkin's lymphoma, multiple myeloma, B-cell lymphoma (e.g., diffuse large B-cell lymphoma (DLBCL)), chronic lymphocytic lymphoma (CLL), T-cell lymphoma, hairy cell lymphoma, and Burkett's lymphoma. Example leukemias include acute lymphocytic leukemia (ALL), acute myelogenous leukemia (AMIL), chronic lymphocytic leukemia (CLL), and chronic myelogenous leukemia (CML).

Other cancers treatable by the administration of the compounds of the invention include liver cancer (e.g., hepatocellular carcinoma), bladder cancer, bone cancer, glioma, breast cancer, cervical cancer, colon cancer, endometrial cancer, epithelial cancer, esophageal cancer, Ewing's sarcoma, pancreatic cancer, gallbladder cancer, gastric cancer, gastrointestinal tumors, head and neck cancer (upper aerodigestive cancer), intestinal cancers, Kaposi's sarcoma, kidney cancer, laryngeal cancer, liver cancer (e.g., hepatocellular carcinoma), lung cancer, prostate cancer, rectal cancer, skin cancer, stomach cancer, testicular cancer, thyroid cancer, and uterine cancer.

In some embodiments, the cancer treatable by administration of the compounds of the invention is multiple myeloma, DLBCL, hepatocellular carcinoma, bladder cancer, esophageal cancer, head and neck cancer (upper aerodigestive cancer), kidney cancer, prostate cancer, rectal cancer, stomach cancer, thyroid cancer, uterine cancer, and breast cancer.

Exemplary cancers, whose growth can be inhibited using the compound of Formula I or a pharmaceutically acceptable salt thereof, or a unit dosage form thereof as disclosed herein, include solid tumors. For example, in some embodiments, the cancer is a lung cancer, e.g., a squamous cell carcinoma of the lung (SCCL or NSCLC). In some embodiments, the cancer is advanced squamous non-small cell lung carcinoma. In some embodiments, the cancer is head and neck squamous cell carcinoma (HNSCC). In some embodiments, the cancer is an esophageal cancer. In other embodiments, the cancer is a breast cancer, e.g., an HR+ breast cancer. In some embodiments, the cancer is ovarian cancer. In other embodiments, the cancer is pancreatic cancer. In certain embodiments, the cancer is one in which PARP7 expression is amplified.

In some embodiments, the subject is administered a dosage regimen disclosed herein until the cancer has progressed. In some embodiments, the subject is administered a dosage regimen disclosed herein until adverse effects are no longer tolerated. In some embodiments, the subject is administered a dosage regimen disclosed herein until the subject withdraws consent to continued treatment. In some embodiments, the subject is administered a dosage regimen disclosed herein until the cancer has been determined to be in remission. “Remission” is defined as a decrease in or disappearance of signs and symptoms of the cancer. In some embodiments, the subject is administered a dosage regimen disclosed herein for one or more treatment cycles of about 21 days.

### Combination Therapy

The compound of Formula I, or a pharmaceutically acceptable salt thereof, or a dosage form containing the same, may be used alone to inhibit the growth of cancerous tumors or may be used in combination with one or more of: a standard of care treatment (e.g., typical for the type of cancer being treated), an antibody or antigen-binding fragment thereof, an immunomodulator (e.g., an activator of a costimulatory molecule or an inhibitor of an inhibitory molecule); a vaccine, e.g., a therapeutic cancer vaccine; or other forms of cellular immunotherapy.

By “a combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. In will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

Further provided herein are methods for the treatment of cancer in a subject in need thereof by administering to the subject (i) the compound of Formula I, or a pharmaceutically acceptable salt thereof, and (ii) an antibody that binds to human PD-1.

In some embodiments, the antibody is a humanized antibody. In some embodiments, the antibody is pembrolizumab. Pembrolizumab is a humanized antibody that is approved by the U.S. Food and Drug Administration to treat melanoma, non-small cell lung cancer (NSCLC), head and neck squamous cell cancer (HNSCC), classical Hodgkin lymphoma (cHL), primary mediastinal B-cell lymphoma (PMBCL), urothelial carcinoma, cancer that is a microsatellite instability-high (MSI-H) or a mismatch repair deficient (dMMR) solid tumor, colon or rectal cancer, gastric cancer or gastroesophageal junction (GEJ) adenocarcinoma, esophageal cancer or certain gastroesophageal junction (GEJ) carcinomas, cervical cancer, hepatocellular carcinoma, Merkel cell carcinoma (MCC), renal cell carcinoma (RCC), cutaneous squamous cell carcinoma (cSCC), and triple-negative breast cancer (TNBC).

The antibody can be administered to a subject, e.g., a subject in need thereof, for example, a human subject, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some embodiments, the antibody is administered by infusion. In some embodiment, the antibody is administered by infusion over a period of less than an hour. In some embodiment, the antibody is administered by infusion over a period of about 10 minutes to about an hour. In some embodiment, the antibody is administered by infusion over a period of about 15 minutes to about 45 minutes. In some embodiment, the antibody is administered by infusion over a period of about 30 minutes.

In some embodiments, the compound of Formula I, or a pharmaceutically acceptable salt thereof, and the antibody are administered simultaneously. In some embodiments, the compound of Formula I, or a pharmaceutically acceptable salt thereof, and the antibody are administered simultaneously or sequentially. In some embodiments, the compound of Formula I, or a pharmaceutically acceptable salt thereof, and the antibody are administered simultaneously. In some embodiments, the compound of Formula I, or a pharmaceutically acceptable salt thereof, and the antibody are administered sequentially.

The compound of Formula I and its pharmaceutically acceptable salts can be administered to a subject, e.g., a subject in need thereof, for example, a human subject, by a variety of methods. In some embodiments, the route of administration is oral. In some embodiments, the compound of Formula I, or a pharmaceutically acceptable salt thereof, is administered as a pharmaceutical composition.

In some embodiments, the compound of Formula I, or a pharmaceutically acceptable salt thereof, is administered once daily. In some embodiments, the compound of Formula I, or pharmaceutically acceptable salt thereof, is administered twice daily.

In some embodiments, the patient is administered about 25 mg, about 50 mg, about 100 mg, about 200 mg, about 300 mg, about 400 mg, or about 500 mg of the compound of Formula I, or a pharmaceutically acceptable salt thereof, measured as the free base, twice daily. In some embodiments, the patient is administered about 100 mg or about 200 mg of the compound of Formula I, or a pharmaceutically acceptable salt thereof, measured as the free base, twice daily. In some embodiments, the patient is administered about 200 mg of the compound of Formula I, or a pharmaceutically acceptable salt thereof, measured as the free base, twice daily. In some embodiments, the patient is administered about 100 mg of the compound of Formula I, or a pharmaceutically acceptable salt thereof, measured as the free base, twice daily.

In some embodiments, the patient is administered about 25 mg, about 50 mg, about 100 mg, about 200 mg, about 300 mg, about 400 mg, or about 500 mg of the compound of Formula I, or a pharmaceutically acceptable salt thereof, measured as the free base, once daily. In some embodiments, the patient is administered about 100 mg or about 200 mg of the compound of Formula I, or a pharmaceutically acceptable salt thereof, measured as the free base, once daily. In some embodiments, the patient is administered about 100 mg of the compound of Formula I, or a pharmaceutically acceptable salt thereof, measured as the free base, once daily. In some embodiments, the patient is administered about 200 mg of the compound of Formula I, or a pharmaceutically acceptable salt thereof, measured as the free base, once daily.

In some examples, the compound of Formula I, or a pharmaceutically acceptable salt thereof, is administered according to a continuous dosing schedule. For instance, the continuous dosing schedule includes administering the compound, or a pharmaceutically acceptable salt thereof, to the subject for two or more consecutive days. In some embodiments, the continuous dosing schedule includes administering the compound, or a pharmaceutically acceptable salt thereof, to the subject for five or more consecutive days. In other embodiments, the continuous dosing schedule includes administering the compound, or a pharmaceutically acceptable salt thereof, to the subject for ten or more consecutive days. In some embodiments, the continuous dosing schedule includes administering the compound, or a pharmaceutically acceptable salt thereof, to the subject for fifteen or more consecutive days. In some examples, the continuous dosing schedule includes administering the compound, or a pharmaceutically acceptable salt thereof, to the subject for twenty or more consecutive days. In some embodiments, the continuous dosing schedule includes administering the compound, or a pharmaceutically acceptable salt thereof, to the subject for twenty-one or more consecutive days.

The anti-PD-1 antibody can be administered to a subject, e.g., a subject in need thereof, for example, a human subject, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection.

The route and/or mode of administration of the antibody can also be tailored for the individual case, e.g., by monitoring the subject, e.g., using tomographic imaging, e.g., to visualize a tumor.

The antibody can be administered as a fixed dose, or in a mg/kg dose. The dose can also be chosen to reduce or avoid production of antibodies against the anti-PD-1 antibody. Dosage regimens are adjusted to provide the desired response, e.g., a therapeutic response or a combinatorial therapeutic effect. Generally, doses of the anti-PD-1 antibody (and optionally a second agent) can be used in order to provide a subject with the agent in bioavailable quantities.

Dosage unit form or “fixed dose” or “flat dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion. An exemplary fixed dose includes 200 mg.

An anti-PD-1 antibody dose can be administered, e.g., at a periodic interval over a period of time (a course of treatment) sufficient to encompass at least 2 doses, 3 doses, 5 doses, 10 doses, or more, e.g., once or twice daily, or about one to four times per week, or preferably weekly, biweekly (every two weeks), every three weeks, monthly. In some embodiments, the antibody is administered once every three weeks. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

An exemplary dosing regimen comprises administration of an anti-PD-1 antibody at a fixed dose of 200 mg once every 3 weeks. In some embodiments, dosing regimen comprises administration of an anti-PD-1 antibody at a fixed dose of about 200 mg once every 3 weeks.

In some embodiments, the term “about” refers to plus or minus 10% of the value. A skilled person in the art would know that the values presented herein can vary due to the conditions of the experiments such as variability in data collection or instruments.

In some embodiments, the cancer is a breast cancer, a cancer of the central nervous system, an endometrium cancer, a kidney cancer, a large intestine cancer, a lung cancer, an oesophagus cancer, an ovarian cancer, a pancreatic cancer, a prostate cancer, a stomach cancer, a head and neck cancer, an urinary tract cancer, a colon cancer, or a cancer in which PARP7 expression is amplified. In some embodiments, the lung cancer is squamous cell carcinoma of the lung.

In some embodiments, the subject has received prior treatment for cancer. In some embodiments, the prior treatment is a platinum doublet chemotherapy or an immune checkpoint blockade inhibitor. In some embodiments, the prior treatment is a platinum doublet chemotherapy. In some embodiments, the prior treatment is an immune checkpoint blockade inhibitor.

In some embodiments, the prior treatment is a platinum doublet chemotherapy and an immune checkpoint blockade inhibitor. In some embodiments, the subject was treated with the platinum doublet chemotherapy and the immune checkpoint blockade inhibitor sequentially. In some embodiments, the subject was treated with the platinum doublet chemotherapy and the immune checkpoint blockade inhibitor in combination.

Exemplary immune checkpoint blockade inhibitors include anti-PD-1/anti-PD-L1 and anti-cytotoxic T-lymphocyte-associated antigen 4 (CTLA-4) inhibitors. In some embodiments, the immune checkpoint blockade inhibitor comprises anti-PD-1/anti-PD-L1 or anti-cytotoxic T-lymphocyte-associated antigen 4 (CTLA-4) inhibitors. In some embodiments, the immune checkpoint blockade inhibitor comprises anti-PD-1/anti-PD-L1 and anti-cytotoxic T-lymphocyte-associated antigen 4 (CTLA-4) inhibitors.

In some embodiments, the subject has failed at least one previous treatment. In some embodiments, the cancer is refractory.

In some embodiments, the subject has a central nervous system metastasis, wherein the subject has a baseline MRI after CNS-directed therapy that shows no evidence of progression, the subject is neurologically stable, and the subject is not on corticosteroid therapy. In some embodiments, the subject has the baseline MRI within 28 days of treatment.

The invention will be described in greater detail by way of specific examples. The following examples are offered for illustrative purposes, and are not intended to limit the invention in any manner. Those of skill in the art will readily recognize a variety of non-critical parameters, which can be changed or modified to yield essentially the same results. It is appreciated that certain features of the invention, which are, for clarity, described in the context of separate embodiments, can also be provided in combination in a single embodiment. Conversely, various features of the invention, which are, for brevity, described in the context of a single embodiment, can also be provided separately or in any suitable sub-combination.

### EXAMPLES

### Example A: Dose Escalation Study of the Compound of Formula I

### Methodology

This protocol is a first-in-human, Phase 1, multi-center, open-label, study designed to evaluate the safety profile, including any dose-limiting toxicities (DLTs), and (maximum tolerated dose) MTD of the compound of Formula I administered as a single agent PO and establish the recommended phase 2 dose (RP2D) of the compound of Formula I. The study has a Dose Escalation Phase, which includes a relative bioavailability assessment, followed by a Dose Expansion Phase in patients with selected tumor types.

In the Dose Escalation Phase of the study, patients with any histologically- or cytologically-confirmed solid tumor type (which may include “solid” lymphoma [e.g., mantle cell]) that is refractory to standard therapy and for whom no therapy exists that would be curative or might provide clinical benefit and therefore for whom experimental therapy is a reasonable option are eligible. In the Dose Expansion Phase of the study, the RP2D will be investigated in the following cancer types: squamous cell carcinoma of the lung (SCCL), head and neck squamous cell carcinoma (HNSCC), hormone receptor positive (HR+) breast cancer, and PARP7 amplified cancer.

Secondary objectives include characterization of the safety and PK profile of the compound of Formula I, evaluation of the bioavailability of a tablet manufactured with a micronized form of the compound of Formula I relative to a nonmicronized form of the compound of Formula I (standard tablet), and identification of preliminary antitumor activity. Biomarkers and their correlation with response to the compound of Formula I and other outcomes will be examined.

The standard tablet of the compound of Formula I is manufactured with a nonmicronized form of the compound of Formula I and will be used in all dose escalation cohorts prior to the evaluation of relative bioavailability of the micronized tablet versus the standard tablet. Micronized tablets will be used in the Dose Expansion Phase of the study after the relative bioavailability assessment has been completed.

### Dose Escalation Phase

This includes evaluating escalating dose levels of the compound of Formula I in cohorts of 3 to 6 patients and a separate cohort(s) to assess the relative bioavailability of micronized and nonmicronized tablets.

Two schedules are employed in the Dose Escalation Phase:
Schedule 1 will examine a 21-day cycle with 14 days of the compound of Formula I administered at doses of 25 mg, 50 mg, 100 mg, 200 mg, 300 mg, and 500 mg BID followed by a rest period.
Schedule 2 will examine a 21-day continuous dosing cycle at doses of 100 mg, 200 mg, 300 mg, and 400 mg BID.

Cohorts will follow a traditional 3+3 design.

The initial patient cohort will be assigned to Schedule 1 and receive the compound of Formula I at a dose of 25 mg twice daily (BID) for 14 days.

Schedule 2 will be initiated after at least one dose-escalation in Schedule 1, as determined by the Safety Review Committee (SRC). Schedule 2 patients will receive the compound of Formula I BID for 21 days at a dose at least one dose level below that being investigated in the active Schedule 1 cohort. Doses will be escalated as determined by Schedule 1 and 2 findings.

To obtain accurate PK estimations, in Schedule 1 the compound of Formula I will be administered once daily (QD) on Cycle 1, Day 1 (C1D1), and then on a BID schedule through C1D14 with a single dose administered in the clinic on C1D15 for an 8-hour PK assessment (replaces the skipped C1D1 evening dose). For Schedule 2, the compound of Formula I will be administered QD on C1D1, and then on a BID schedule through C1D21 with an 8-hour PK assessment on C1D15. For the purposes of this study, a treatment cycle is 21 days. (C1D21 is the last day of C1; the first day of C2 is C2D1, equivalent to study Day 22). Based on PK data from BID dose cohorts in this study, QD, three times daily (TID) dosing (either intermittent or continuous), or other schedules also may be explored.

The first study drug dose will be administered on C1D1, with the exception of the Relative Bioavailability Cohort(s), described below, in which the first study drug dose will be given 7 days (±3 days) prior to C1D1. Each patient in a dose cohort must have received at least 85% of their prescribed compound of Formula I doses, unless due to treatment-related toxicity (by default, a DLT), during C1 with follow-up safety evaluations through C2D1 to be eligible for the assessment of DLT. Patients who are not eligible for assessment of DLT will be replaced.

### Relative Bioavailability

Relative Bioavailability Cohort(s) of approximately 6 to 8 patients will be enrolled to evaluate the relative bioavailability of a tablet manufactured with a micronized form of the compound of Formula I (micronized tablet) compared to a nonmicronized form of the compound of Formula I (standard tablet). Patients will receive the first dose of study drug, administered as 100 mg of the micronized tablet, 7 days (±3 days) prior to C1D1. After a minimum washout period of 4 days, patients will receive the second dose of study drug, administered as 100 mg of the standard (unmicronized) tablet, on C1D1. Assessments conducted after C1D1 will be the same as described for patients in the dose escalation cohorts following Schedule 2. Based on emerging PK data, additional Relative Bioavailability Cohorts at additional dose levels may be explored. After a review of the PK and safety results from the Relative Bioavailability Cohort(s), the micronized tablet may be used in subsequent dose escalation and/or dose confirmatory cohorts. Micronized tablets will be used in the Dose Expansion Phase of the study after the relative bioavailability assessment has been completed.

Patients enrolled in the Relative Bioavailability Cohort(s) will have an additional visit 7 days (±3 days) prior to C1D1. Patients are to present to the study center before the first study drug dose, administered as the micronized tablet, on C1D-7 and are to remain at the study center through at least 12 hours after the first study drug dose. On C1D-7 patients will be attached to a Holter monitor that will run continuously for ˜ 24 hours (i.e., until patients return on the following day for the 24-hr PK sample collection on C1D-6). Patients will return to the study center for the second study drug dose, administered as the standard tablet, on C1D1 and are to remain at the study center through at least 12 hours after the second study drug dose. On C1D1, patients will be attached to a Holter monitor that will run continuously for ˜24 hours (i.e., immediately before the study drug dose on C1D2). Patients are to return the following day (C1D2) to receive study drug (administered as the standard tablet) 24 hours (+1 hour) after the study drug dose on C1D1, and immediately after the 24-hour PK sample collection. Assessment of relative bioavailability will be made based upon the comparison of PK data collected in the 24 hour periods after dosing on C1D-7 and C1D1. Samples collected after C1D2 are not considered part of the relative Bioavailability data set. Patients will continue to receive the standard tablet for all subsequent doses, and assessments conducted after C1D1 will be the same as described for patients in the dose escalation cohorts following Schedule 2.

### Dose Expansion Phase

Four cohorts of approximately 20 patients each will be enrolled to further examine the safety, PK, pharmacodynamics, and antitumor activity of the Compound of Formula I at the RP2D. Based on nonclinical data as well as clinical data obtained from the Dose Escalation Phase of this study, enrollment of patients in the Dose Expansion Phase will be limited to the following specific tumor type(s): SCCL, HNSCC, HR+ breast cancer, and PARP7 amplified cancer. Each cohort will enroll approximately 20 patients and the sample size may be expanded to approximately 40 patients, if at least 3 patients out of the 20 treated have an objective response (PR or CR) per RECIST 1.1 in that group. If enrolling 20 patients to any one of these cohorts is not logistically feasible, then that cohort may be closed to enrollment at any time.

### Study Drug Supply

The compound of Formula I will be supplied as 25 mg, 100 mg, and 200 mg standard tablets for oral administration. A tablet manufactured with a micronized form of the compound of Formula I will be supplied as 100 mg tablets for oral administration. Particle diameter size [d90] will be 5-15 μm for the micronized compound.

### Data

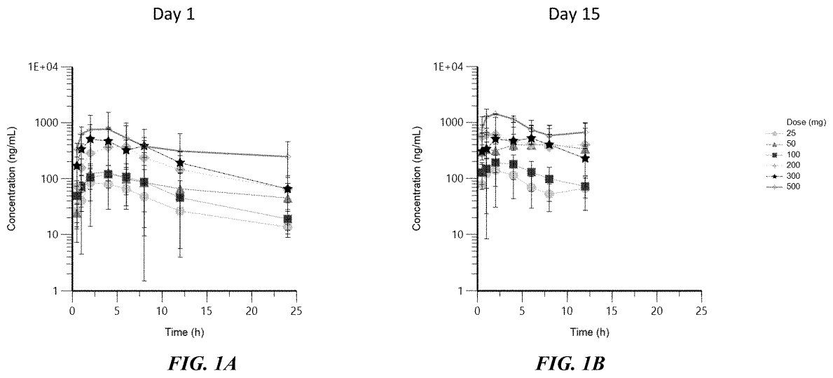

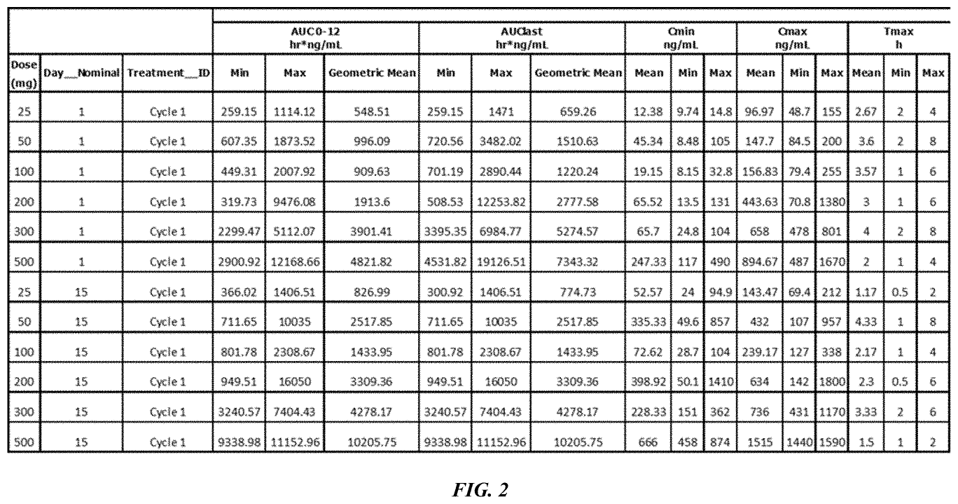

Initial human PK data has been obtained with respect to dosing nonmicronized compound of Formula I (d[90] 228.474 μm) which is shown in FIGS. 1-2.

Additional PK data has been obtained comparing micronized (d[90] 10.851 μm) with nonmicronized (d[90] 228.474 μm) compound of Formula I. Patients were administered 100 mg of the compound of Formula I as a micronized tablet. Plasma samples were collected at 0.5, 1, 2, 4, 6, 8, 12 and 24 hours post dose. After a seven day washout period, patients were administered 100 mg of the compound of Formula I as a nonmicronized tablet. Plasma samples were collected at 0.5, 1, 2, 4, 6, 8, 12 and 24 hours post dose. Area under the curve and maximum plasma concentration data were derived via non-compartmental analysis for the micronized and nonmicronized dosing arms. See Table below. The mean AUC increased by about 1.5-fold and the mean Cₘₐₓ increased by about 2.4-fold.

| | | | AUCₗₐₛₜ | | Cₘₐₓ | |
|---|---|---|---|---|---|---|
| | | | h*ng/mL | | ng/mL | |
| | | | Geometric | Geometric | Geometric | Geometric |
| Treatment | Dose | N | Mean | SD | Mean | SD |
| Micronized | 100 mg | 7 | 4471 | 1.92 | 803 | 1.91 |
| Nonmicronized | 100 mg | 7 | 3007 | 1.83 | 337 | 1.62 |

### Example B: Pharmacokinetics of the Compound of Formula I Following Oral Administration of Tablets and Capsules in Male Beagle Dogs

### Objective

The objective of this study was to determine the plasma pharmacokinetic profiles of the compound of Formula I in male Beagle dogs after oral administration and to assess the exposure of tablets and capsules formulations.

### Animals

Male Beagle dogs were purchased from Jiangsu Johnsen Bioresource CO., Ltd. The animals were 1-2.5 years old on the dosing day. All animals were housed in a 12-hour light/12-hour dark cycle environment. Animals for oral (PO) groups were fasted overnight prior to dosing and were fed approximately 2 hours after dosing. This study was approved by the Pharmaron Institutional Animal Care and Use Committee.

### Study Design

A total of five male Beagle dogs were dosed via a design as shown in Table 1 below. The compound of Formula I was used for the dose formulation and standard stock solution preparation. Compound of Formula I was administered as tablet and capsule doses. For the animals in PO groups, pentagastrin (6.0 μg/kg, i.m.) was administrated 20 minutes before dosing PO formulation and 1.5 hours after the first pentagastrin dosing. The dosing volume was 0.024 mL/kg, the concentration was 250 μg/mL in DMSO/1 N NaOH/PBS. The dosing volume, detailed dosing time of pentagastrin was recorded. 10 mL of 0.001 N HCl was used to wash the gavage catheter for each animal.

TABLE 1
Dosing information

| | | Dose Level | Dose | No. of |
|---|---|---|---|---|
| Group | Treatment | (mg/dog) | Route | Animals |
| A | Tablet | 25 | PO | 5 males |
| B | Tablet | 50 | | |
| C | Tablet | 100 | | |
| D | Capsule: | 100 | | |
| | Nonmicronized | | | |
| E | Capsule: Micronized | 100 | | |

### Formulation Preparation

Preparation of pentagastrin intramuscular formulation (6.0 μg/kg) Pentagastrin was dissolved in DMSO/1 N NaOH/physiologically buffered saline (1/2/300, v/v/v) at a concentration of 250 μg/mL. The weight of each capsule dose containing Compound of Formula I was recorded, the weights of pentagastrin and 1-ABT were recorded. Tablets each containing 25 mg of Compound of Formula I were supplied on behalf of the Sponsor.

Capsules were filled with appropriate amount and kind (nonmicronized and micronized) of the compound of Formula I.

### Sample Collection

For PO (25, 50, 100, 100, 100 mg/dog) administration, blood samples were collected from each animal at Pre-dose, 15 min, 30 min, 1, 2, 4, 8, 24 hours post dose. Blood samples (0.5 mL) were collected from each animal via jugular veins. These blood samples were placed into tubes containing dipotassium ethylenediaminetetraacetic acid and then centrifuged at 2000 g for 10 minutes at 4° C. to produce plasma. All samples were stored at −75±15° C. until analysis.

### Preparation of Standard Solutions for LC-MS/MS Analysis

Compound of Formula I was prepared in dimethyl sulfoxide with vortex at 1 mg/mL (free form) as standard stock solution. Calibration standard working solutions were prepared at concentrations of 5, 10, 20, 50, 100, 500, 1000, 5000, 10000 and 20000 ng/mL by serial dilution of the standard stock solution by 50% acetonitrile in water. Quality control working solutions at concentrations of 10, 20, 50, 1000, 8000 and 16000 ng/mL were prepared by serial dilution of the standard stock solution by 50% acetonitrile in water. These QC samples were prepared on the day of analysis in the same way as calibration standards.

### Sample Treatment

5 μL of each calibration standard working solution (5, 10, 20, 50, 100, 500, 1000, 5000, 10000 and 20000 ng/mL) was added to 50 μL of blank Beagle Dog plasma to achieve calibration standards of 0.5-2000 ng/mL (0.5, 1, 2, 5, 10, 50, 100, 500, 1000, 2000 ng/mL) in a total volume of 55 μL. Quality Control (QC) samples at 1 ng/mL (low-1), 2 ng/mL (low-2), 5 ng/mL (low-3), 100 ng/mL (mid), 800 ng/mL (high-1), 1600 ng/mL (high-2) in blank plasma were prepared independently from those used for the calibration curves. These QC samples were prepared on the day of analysis in the same way as calibration standards. 55 μL of standards, 55 μL of QC samples or 55 μL of unknown samples (50 μL of plasma sample with 5 μL 50% acetonitrile in water) were mixed with 200 μL of acetonitrile containing IS (internal standard, dexamethasone) to precipitate proteins. Then the samples were vortexed for 30 sec. After centrifugation at 4° C., 3900 rpm for 15 min, the supernatant was diluted 7 times with water, 1 μL supernatant was injected into the LC-MS/MS system for quantitative analysis.

### Data and Statistical Analysis

Data acquisition was performed by Sciex Analyst 1.6.3 software (AB Sciex, Forster City, Calif.). Pharmacokinetic parameters were calculated by non-compartmental analysis (Phoenix™ WinNonlin© 6.1). The linear trapezoidal algorithm was used for AUC calculation.

### Pharmacokinetic Analysis/Summary

The pharmacokinetics of the compound of Formula I were evaluated in male Beagle dogs following oral (PO) administration. PO administration was conducted with Compound of Formula I formulated as tablets and micronized/nonmicronized compound of Formula I formulated as gelatin capsules.

The oral exposure of dogs in Groups A, B and C increased with the dose level, though not linear. The exposure of dogs in E group was much higher than dogs in Group D, which were administrated with micronized and nonmicronized compound of Formula I capsules, respectively. Summary of PK results are shown in Table 2 below.

TABLE 2
Mean Plasma Pharmacokinetic Parameters of the compound of Formula I Following
PO (25, 50, 100, 100, 100 mg/dog) Administration of Tablets and Capsule

| | | Dose | | | | | | | |
|---|---|---|---|---|---|---|---|---|---|
| | | level | t_{1/2} | Tₘₐₓ^{#} | Cₘₐₓ | AUC_{(0-24 h)} | AUC_{(0-∞)} | MRT_{inf} | AUC_{(0-24 h)}/D |
| Group | Treatment | (mg/dog) | (h) | (h) | (ng/mL) | (h*ng/mL) | (h*ng/mL) | (h) | (h*mg/mL) |
| A | Tablet | 25 | 3.9 | 1.0 | 371 | 1983 | 2015 | 5.0 | 836 |
| B | Tablet | 50 | 4.8 | 1.0 | 489 | 3120 | 2968 | 5.5 | 664 |
| C | Tablet | 100 | 4.0 | 2.0 | 691 | 4214 | 4371 | 5.1 | 444 |
| D | Capsule: | 100 | 4.2 | 2.0 | 356 | 3398 | 3415 | 6.1 | 360 |
| | Standardized | | | | | | | | |
| E | Capsule: | 100 | 4.3 | 2.0 | 1671 | 9565 | 9772 | 5.5 | 1001 |
| | Micronized | | | | | | | | |

NA—not applicable
^{#}Tₘₐₓ median aggregation.
NV = no value as median result is not at a defined sample time point.

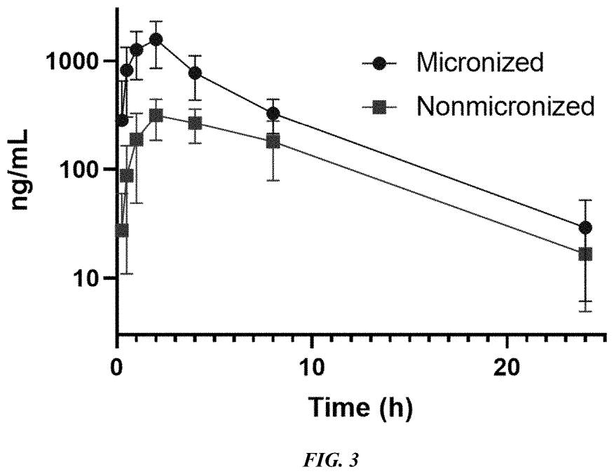

The micronization of the Compound of Formula I improved exposure in dogs. Micronization of the Compound of Formula I resulted in a 2.8-fold increase in AUC and a 4.7-fold increase in Cₘₐₓ. Data is shown below in Table 3 and in FIG. 3.

TABLE 3
Characterization of micronized Compound of Formula I in dogs

| Particle Size | Cₘₐₓ | AUC |
|---|---|---|
| (d90:d50 μm) | (ng/mL) | (ng*h/mL) |
| 228:108 | 356 | 3398 |
| 18:5 | 1671 | 9565 |

### Example C: Tablets Comprising the Compound of Formula I

Tablets for oral administration in the clinical study described in Ex. A containing the compound of Formula I were prepared by wet granulation. Table 4 below provides exemplary tablet compositions prepared accordingly. The 100 mg tablet was prepared with either micronized or nonmicronized compound of Formula I.

TABLE 4

| | 25 mg | 100 mg | 200 mg | |
|---|---|---|---|---|
| | (mg/ | (mg/ | (mg/ | |
| Component | tablet) | tablet) | tablet) | Function |
| Compound of Formula I | 25.0 | 100.0 | 200.0 | Active Ingredient |
| Microcrystalline | 12.5 | 50.0 | 100.0 | Filler/Diluent |
| cellulose | | | | |
| Mannitol | 20.0 | 80.0 | 160.0 | Filler/Diluent |
| Crospovidone XL | 2.6 | 10.0 | 20.0 | Disintegrant |
| Hydroxypropyl cellulose | 1.3 | 5.0 | 10.0 | Binder |
| Sodium stearyl fumarate | 1.3 | 5.0 | 10.0 | Lubricant |
| TOTAL | 62.5 | 250 | 500 | |

### Example D: Micronized Compound of Formula I

Micronized batches of the compound of Formula I were prepared by jet milling using a nitrogen gas feed. The nonmicronized starting material was prepared in a manner similar to the process for preparing crystalline compound of Formula I described in U.S. Pat. No. 10,550,105. Particle size was measured by laser diffraction according to USP 42 <429> using a Malvern Mastersizer 3000 equipped with a Hydro MV wet dispersion sample unit. Particle size diameters [d90] for two different lots were measured as 10.851 and 8.715 μm, respectively.

### Example E: Clinical Study Protocol for a Phase 1b/2, Multicenter, Single Arm Study of the Compound of Formula I in Combination with Pembrolizumab in Patients with Squamous Cell Carcinoma of the Lung (SCCL)

### Objective

The objective of this study in the Phase 1b Safety Run-in is to determine the recommended Phase 2 dose of the compound of Formula I (RP2D) in combination with pembrolizumab.

The objective of this study in Phase 2 is to evaluate the antitumor activity of the Compound of Formula I in combination with pembrolizumab in SCCL patients with secondary resistance to prior immune checkpoint blockade inhibitor (ICI) treatment.

Secondary objectives include to assess additional measures of antitumor activity, to evaluate the safety profile and tolerability of the compound of Formula I in combination with pembrolizumab, and to characterize the pharmacokinetic (PK) properties of the compound of Formula I.

Exploratory objectives include to evaluate the predictive biomarkers that may correlate with response and to characterize pharmacodynamics (Pd) effects of the compound of Formula I in combination with pembrolizumab.

### Study Population

The study will enroll male and female patients≥18 years of age with a confirmed diagnosis of advanced non-small cell lung carcinoma (NSCLC) of squamous cell histology that have received prior therapy, including a platinum doublet, and an approved ICI either sequentially or in combination.

### Number of Patients (Planned)

Approximately 50 patients are planned for enrollment in this study: Phase 1b Safety Run-in: up to 10 patients; Phase 2: approximately 40 patients.

### Study Design

This is an open label, multicenter Phase 1b/2 single-arm study that will evaluate the safety and antitumor activity of the compound of Formula I in combination with pembrolizumab in patients with confirmed diagnosis of advanced NSCLC of squamous cell histology who have received prior therapy. The study consists of a Phase 1b Safety Run-in and Phase 2. The Phase 1/b Safety Run-in and Phase 2 will involve 4 study-conduct periods: Screening Period, On-Treatment Period, End-of-Study Period (30-day safety follow-up), and Follow-up Period (90-day follow-up for assessment of immune-related adverse events [irAEs] and disease progression).

During the Phase 1b Safety Run-in, up to 10 patients determined to be eligible for treatment during Screening will be enrolled in a 3+3 fashion and will initiate treatment with continuous oral dosing with 200 mg of the compound of formula I twice daily (BID) in combination with the fixed approved dose of intravenous (IV) pembrolizumab (200 mg every 3 weeks [Q3W]). A Safety Review Committee comprised of the principal investigators, Medical Monitor, and Sponsor will evaluate all safety data and any available PK data from all treated patients to confirm any dose limiting toxicities (DLTs) that occurred during Cycle 1 of the combination treatment. A dose de-escalation cohort may be opened based on the emerging safety profile to confirm the tolerability of the continuous dose level of the compound of Formula I in combination with pembrolizumab and to confirm the RP2D.

During Phase 2, approximately 40 patients determined to be eligible for treatment during Screening will initiate treatment with continuous oral dosing with the RP2D of the compound of Formula I BID in combination with the fixed approved dose of IV pembrolizumab. Patients from the Phase 1b Safety Run-in who were treated at the RP2D will also contribute to the Phase 2 sample size.

In both the Phase 1b Safety Run-in and Phase 2, a treatment cycle is defined as 21 days.

On Day 1 of each treatment cycle, the compound of Formula I will be administered orally and followed by IV infusion of pembrolizumab at the fixed approved dose according to the approved local product label. During the remaining days of the 21-day treatment cycle, only the compound of Formula I BID will be administered. Treatment cycles will continue until disease progression, unacceptable treatment-related toxicity, or withdrawal of consent.

If a patient discontinues treatment with the compound of Formula I, the patient will be discontinued from the study and will be followed-up for safety and progression free survival (PFS).

Patients will remain on-treatment with the compound of Formula I single agent if there are irAEs due to the administration of pembrolizumab. A patient who discontinues treatment of the combination before occurrence of progressive disease (PD) will be followed off-treatment until PD is documented, the patient withdraws consent for further follow-up, or at the start of new antineoplastic therapy.

Treatment with the combination or with the compound of Formula I single agent may be continued in the presence of radiographic progression if, after discussion between the investigator and the sponsor, it is determined that a patient would derive benefit from continued therapy.

Periodic safety assessments will include routine safety laboratory assessments, physical examinations, 12-lead electrocardiograms (ECGs), and vital signs. Continual safety assessment of treatment-emergent adverse events (TEAEs), irAEs, and concomitant medications will be conducted. Computed tomography (CT) and/or magnetic resonance imaging (MRI) will be used to assess the response of disease to treatment. Periodic blood sampling will be used to characterize Pd and PK parameters. Exploratory biomarkers will be assessed through blood and biopsy samples.

### Eligibility Criteria

A patient must meet all of the following criteria to be eligible for this study:
1. Male or female patients (i) 18 years or older and (ii) 99 years or younger.
2. Confirmed diagnosis of advanced/metastatic NSCLC of squamous cell histology as determined by local testing practices:
   a. Patients should have received prior therapy including a platinum doublet and an ICI, including anti-PD-1/anti-PD-L1, anti-cytotoxic T-lymphocyte-associated antigen 4 (CTLA-4) inhibitors, either sequentially or as combination of chemo+checkpoint inhibitor.
      and
   b. The last regimen prior to enrolling in the study must be an approved checkpoint inhibitor-containing regimen where the best response was stable disease, partial response or complete response.
   Note: Combination of anti-PD-1/anti-PD-L1 and anti-CTLA-4 are considered one line of therapy.
3. Patients experienced PD as determined by the investigator during or following their most recent treatment regimen. Supporting information about PD must be documented.
4. Patient will have provided signed and dated informed consent prior to initiation of any study procedures.
5. Patient agrees to undergo biopsy for fresh tissue sample collection, provided the procedure to obtain the biopsy is deemed a non-significant risk procedure and is safe to do so. Patient consent for tumor biopsy collection (during Screening) is mandatory.
   a. Archival biopsy samples collected within 12 weeks prior to Screening are acceptable as the baseline biopsy.
   b. In the event that collection of a fresh biopsy is not medically feasible or in cases where the fresh biopsy is deemed not evaluable (e.g., no tumor present), submission of an archival sample collected >12 weeks prior to Screening may be requested.
6. Eastern Cooperative Oncology Group (ECOG) performance status of 0 to 1.
7. Have CT or MRI imaging done within 28 days prior to study treatment and have at least one measurable target lesion per Response Evaluation Criteria in Solid Tumors (RECIST) version 1.1.
8. Patient has adequate organ function as specified by the following laboratory values:
   a. Patient has adequate renal function (creatinine≤1.5× the upper limit of normal [ULN]) or a glomerular filtration rate (GFR) of ≥60 mL/min/1.73 m²).
   b. Patient has adequate hepatic function, as evidenced by a total bilirubin≤1.5×ULN (unless due to Gilbert's Syndrome who are excluded if total bilirubin>3.0×ULN and direct bilirubin>1.5×ULN), aspartate aminotransferase (AST), and/or alanine aminotransferase (ALT)≤2.5×ULN or ≤5×ULN, if due to liver involvement by tumor.
   c. Patient has adequate bone marrow function, as evidenced by hemoglobin≥9.0 g/dL in the absence of transfusion within the previous 72 hours, platelet count≥100×10⁹ cells/L, and absolute neutrophil count (ANC)≥1.5×10⁹ cells/L. The requirement for an ANC≥1.5×10⁹/L must be independent of recent growth factor support (e.g., within the last 7 days).
9. Patient and his/her partner agree to use adequate contraception after providing written informed consent through 4 months after the last study drug dose, as follows:
   a. For women: Negative pregnancy test during Screening and compliant with a medically approved contraceptive regimen during and for 4 months after the treatment period or documented to be surgically sterile or postmenopausal. The use of hormonal contraception (oral or implanted) is not considered a medically approved regimen, as the compound of Formula I may interfere with the efficacy of hormonal based contraception.
   b. For men: Compliant with a medically approved contraceptive regimen during and for 4 months after the treatment period or documented to be surgically sterile.
10. Patient is willing and able to participate in the study and comply with all study requirements.

### Exclusion Criteria

A patient who meets any of the following criteria will be excluded from this study:
1. Has non-squamous histology NSCLC. Patients whose tumors have a mixed histology are ineligible.
2. Patient must not have received any other investigational systemic therapy for SCCL.
3. Patient should not have received more than two prior lines of therapy with ICI including anti-PD-1/anti-PD-L1, anti-CTLA-4 inhibitors and one prior line of a chemotherapy doublet treatment.
4. Patient is unable to swallow oral medications, has impairment of gastrointestinal (GI) function or GI disease that may significantly alter drug absorption (e.g., active inflammatory bowel disease, uncontrolled nausea, vomiting, diarrhea, or malabsorption syndrome).
5. Prior radiation within 2 weeks of Cycle 1 Day 1 (C1D1), except for palliative radiotherapy to a limited field. Patients must have recovered from all radiation-related toxicities, not require corticosteroids, and not have had radiation pneumonitis. A 1-week washout is permitted for palliative radiation (≤2 weeks of radiotherapy) to non-CNS disease.
6. A patient with CNS metastases is excluded if:
   Has active CNS metastases (new lesions or progression from prior imaging study) requiring treatment within 28 days prior to study treatment and/or ongoing corticosteroid therapy.
   Has symptomatic or untreated leptomeningeal disease.
7. Patients who discontinue prior treatment with an ICI due to irAEs.
8. Has a known history of prior malignancy. Except: malignancies that were treated curatively and have not recurred within 2 years prior to study treatment; completely resected basal cell and squamous cell skin cancers; any malignancy considered to be indolent and that has never required therapy; and completely resected carcinoma in situ of any type.
9. Has received a live-virus vaccination within 30 days of planned treatment start. Vaccines that do not contain live virus are permitted.
10. Any of the following in the previous 6 months: myocardial infarction or current history of New York heart Association (NYHA) Class III or IV heart failure, uncontrolled angina, severe uncontrolled ventricular anemias, or electrocardiographic evidence of acute ischemia.
11. Patient has a history of prolonged QT syndrome or Torsades de pointes, and/or has a familial history of prolonged QT syndrome.
12. Patient is taking a concomitant medication that is a strong inhibitor or inducer of cytochrome P450 [CYP]-mediated metabolism or that is metabolized by CYP 2B6, 3A4 or 2C19, 2C9, or other members of the IIC subfamily of the CYP genes and that, if underdosed, would constitute a significant risk to the patient. Individual cases may be discussed with the Medical Monitor.
13. Ingestion of herbal medicines within 3 weeks before Screening, and grapefruit, grapefruit juice, pomegranate juice, star fruit, or orange marmalade (made with Seville oranges) within 1 week prior to Screening. (Note that there are well-reported cases of CYP3A drug-drug interactions with these foodstuffs.)
14. Has active autoimmune disease that has required systemic treatment in the past 12 months (i.e., with use of disease modifying agents, corticosteroids or immunosuppressive drugs). Replacement therapy (e.g., ≤10 mg daily prednisolone or steroid equivalent, thyroxine, insulin, or corticosteroid replacement therapy for adrenal or pituitary insufficiency, etc.) is not considered a form of systemic treatment. Patients with vitiligo, resolved childhood asthma/atopy, type I diabetes mellitus, and residual hypothyroidism due to an autoimmune condition and only requiring hormone replacement, are not excluded.
15. Is on chronic systemic steroids (e.g., >10 mg daily prednisolone or steroid equivalent for >6 months). Subjects with asthma that require intermittent use of bronchodilators, inhaled steroids, or local steroid injections would not be excluded from the study.
16. Has an active systemic infection requiring therapy (e.g.: bacterial, fungal, viral).
17. Has known active Hepatitis B or C. Active Hepatitis B is defined as a known positive HBsAg result. Active Hepatitis C is defined by a known positive Hepatitis C Antibody result and known quantitative hepatitis C virus ribonucleic acid (RNA) results greater than the lower limits of detection of the assay.
18. Has known psychiatric or substance abuse disorder that would interfere with cooperation with the requirements of the trial.
19. Has interstitial lung disease or a history of pneumonitis that required oral or intravenous steroids to assist with management. Lymphangitic spread of the NSCLC is not exclusionary.
20. Is pregnant or breastfeeding or expecting to conceive or father children while on study medication and for the required duration of contraception after the last dose of study medication.
21. Has ongoing acute clinical AEs of National Cancer Institute (NCI) Common Terminology Criteria for Adverse Events (CTCAE) Grade≥2 resulting from prior cancer therapies (except alopecia, peripheral neuropathy and ototoxicity, which are excluded if ≥Grade 3).
22. Has had, within the past 6 months, the occurrence of one or more of the following events: cerebrovascular accident, deep vein thrombosis, pulmonary embolism, hemorrhage (CTCAE Grade 3 or 4), chronic liver disease (meeting criteria for Child-Pugh Class B or C), organ transplantation.
23. Has, within 2 weeks prior to Day 1, received systemic therapeutic doses of corticosteroids (e.g., >10 mg daily prednisolone or steroid equivalent). Topical, inhaled, nasal and ophthalmic steroids are allowed for short-term treatment of acute conditions (e.g.: asthma, poison ivy contact dermatitis); for other immunosuppressive agents, the exclusionary dose and duration will be determined in consultation with the Medical Monitor.
24. Has any other medical or personal condition that, in the opinion of the Investigator, may potentially compromise the safety or compliance of the patient, or may preclude the patient's successful completion of the clinical study.

### Duration of Treatment and Study Participation

The expected duration of the Phase 1b Safety Run-In assessment period is 1 cycle (3 weeks), and patients will remain on study as per the schedule of events. All patients in the Phase 1b Safety Run in and Phase 2 will remain on-treatment until disease progression, intolerable toxicity, withdrawal of consent, or any other reason according to the investigator's clinical judgment. Patients who discontinue treatment prior to experiencing progression of disease will be followed off-treatment until PD occurs, until the patient withdraws consent for further follow-up, or at the start of new antineoplastic therapy. After consultation with the Medical Monitor, the compound of Formula I as a single agent or in combination with pembrolizumab may be continued for a patient who has met the criteria for PD but, in the Investigator's opinion, is receiving benefit.

The end of the study will occur when all patients have discontinued on-study treatment for any reason, have completed the 30-day or 90-day safety follow up period, have completed the follow-up for disease progression period, or if the study is terminated early.

### Dose Finding: Phase 1b Safety Run-In

During the Phase 1b Safety Run-in, groups of 3 patients will be enrolled in a 3+3 dose-finding design to confirm the tolerability of the compound of Formula I in combination with pembrolizumab at a fixed dose of 200 mg IV every 3 weeks. The first 3 patients will receive the study drug as described below (Selecting a Dose for Each Patient). Upon completion of Cycle 1, the Safety Review Committee will assess the tolerability of the combination according to DLT criteria (Table 5), safety data, and PK data. Depending on the tolerability of the combination, additional groups of 3 patients will be enrolled at the same or a de-escalated dose of the compound of Formula I for further assessment of tolerability after Cycle 1 and for determination of the RP2D. During the Phase 1b Safety Run-in, the occurrence of DLTs (Error! Reference source not found.) will determine the RP2D of the compound of Formula I in combination with pembrolizumab as follows:

After Cycle 1, if ≤1 DLT out of the first 3 patients is determined based on the SRC evaluation and outcome, an additional 3 patients will be treated at the 200 mg of the Compound of Formula I BID dose in combination with pembrolizumab. After Cycle 1, if ≤1 DLT out of the 6 patients treated at that dose is determined based on the SRC evaluation and outcome, the 200 mg of the Compound of Formula I BID dose combination regimen will be declared the RP2D, and the Phase 1b Safety Run-in will be considered complete and the Phase 2 will enroll.

After Cycle 1, if ≥2 DLTs out of 3 patients are determined based on SRC evaluation and outcome, an additional 3 patients will be enrolled at a de-escalated dose of 100 mg of the compound of Formula I BID in combination with pembrolizumab.

After Cycle 1, if ≤1 DLT out of the 3 newly enrolled patients is determined based on SRC evaluation and outcome, then an additional 3 patients will be treated at the 100 mg of the compound of Formula I BID dose in combination with pembrolizumab. After Cycle 1, if ≤1 DLT out of the 6 patients treated at that dose is determined based on SRC evaluation and outcome, then the compound of Formula I 100 mg BID dose in combination with pembrolizumab will be declared the RP2D and the Phase 1b Safety Run-in will be considered complete and the Phase 2 will enroll. If ≥2 DLT out of the 6 patients treated at that dose is determined based on SRC evaluation and outcome, then the 100 mg of the compound of Formula I BID dose in combination with pembrolizumab will be declared not tolerable and the study will not proceed.

After Cycle 1, if ≥2 DLT out of the 3 newly enrolled patients is determined based on SRC evaluation and outcome, then the 100 mg of the compound of Formula I BID dose in combination with pembrolizumab will be declared not tolerable and the study will not proceed.

### Dose Limiting Toxicities

No dose escalation of the compound of Formula I will be permitted during the study. The starting dose of the compound of Formula I may be de-escalated to 100 mg PO BID during the Phase 1b Safety Run-in based on the incidence of DLTs (Table 5).

The most frequent compound of Formula I treatment-related AEs in the ongoing Phase 1 escalation study are dysgeusia or taste disorder (36%), decreased appetite (15%), fatigue (14%), nausea (12%), and anemia, constipation, diarrhea and decreased weight (10%) each.

The most frequent treatment-related AEs observed with pembrolizumab as a single agent (reported in >20%) include fatigue, musculoskeletal pain, decreased appetite, pruritus, diarrhea, nausea, rash, pyrexia, cough, dyspnea, constipation, pain and abdominal pain (Merck 2021). The safety profile in patients with NSCLC that received pembrolizumab as a second or third line of therapy in the Keynote-010 study (n=682) included diarrhea, fatigue, decreased appetite, liver enzyme elevation, pneumonia, and pneumonitis. Adverse reactions leading to discontinuation occurred in 8% of patients with pneumonitis being the most common (1.8%).

Even though pembrolizumab and the compound of Formula I have different clearance mechanisms and no drug-drug interactions (DDIs) would be expected, based on available data, potential overlapping toxicities include fatigue, decreased appetite, and GI symptoms such as nausea, constipation, and diarrhea.

DLTs are presented in Table 5 and are defined as any of the following events in Cycle 1 that are considered by the Investigator to be at least possibly related to the compound of Formula I and/or pembrolizumab. The National Cancer Institute Common Terminology Criteria for Adverse events version 5.0 will be used (NCI CTCAE v5.0).

TABLE 5
Dose-Limiting Toxicities (compound of Formula I)

| Toxicity | Any of the Following Criteria |
|---|---|
| Hematology | Grade 3 neutropenia with fever of ≥38.5° C. |
| | (101.3° F.). |
| | Grade 4 neutropenia persisting for >5 days |
| | Grade 4 thrombocytopenia or Grade 3 thrombocytopenia |
| | either accompanied by clinically significant bleeding |
| | or requiring dose interruption for ≥5 days |
| Hepatobiliary | ≥CTCAE Grade 3 total bilirubin |
| | CTCAE Grade 2 total bilirubin and CTCAE Grade 2 |
| | ALT or AST |
| | ≥CTCAE Grade 3 ALT or AST |
| ECG QT | Corrected QT interval (QTcF interval) ≥501 msec |
| interval | or QTcF variation from the pre-dose baseline that |
| | day >60 msec on ≥2 ECGs performed at |
| | the same visit. |
| Gastrointestinal | ≥CTCAE Grade 3 nausea or vomiting lasting ≥72 |
| | hours despite optimal anti-emetic or supportive therapy |
| | ≥CTCAE Grade 3 diarrhea lasting ≥72 hours |
| | despite optimal anti-diarrhea or supportive therapy |
| | ≥CTCAE Grade 3 nausea, vomiting, or diarrhea |
| | regardless of duration or optimal antiemetic/supportive |
| | care therapy |
| Immune-related | Grade 2 pneumonitis lasting ≥7 days despite optimal |
| | treatment |
| toxicities | Grade 3 pneumonitis |
| | Grade 3 immune-related toxicities that persist for >7 |
| | days despite treatment with corticosteroids |
| | Grade 4 immune-related toxicities of any duration |
| Other AEs | ≥CTCAE Grade 3 toxicities except for the exclusions |
| | noted below |
| | Any other ≥Grade 2 toxicity that in the opinion of |
| | the Investigator and agreed with the Medical Monitor |
| | is a clinically unacceptable risk |
| | Any AE that results in a prolonged delay (>14 days) |
| | in initiating cycle 2 or permanent discontinuation |
| | prior to initiating cycle 2 |
| | Any Grade 5 toxicity |
| Exceptions to | |
| DLT criteria | Grade 3 fatigue that improves to ≤Grade 1 within 5 days |
| | Grade 3 electrolyte disturbance resolving to <Grade |
| | 1 or baseline within 7 days of drug interruption |
| | (supplementation allowed) |

Abbreviations: ALT = alanine transaminase; AST = aspartate transaminase; CTCAE = Common Terminology Criteria for Adverse Events; DLT = dose limiting toxicity; ECG = electrocardiogram; QTcF = QT interval corrected with Fridericia's formula

If there is a previous DLT in a group followed by a Grade 3 irAE in the same or previous cohort, further enrollment and treatment of patients in the cohort should be held for up to 21 days while awaiting the outcome of the Grade 3 irAE. If the Grade 3 irAE does not resolve to ≤Grade 1 within 21 days, it will be considered a DLT and enrollment in this cohort should be stopped.

### Treatment Combination: Compound of Formula I and Pembrolizumab

Nonclinical studies support the clinical evaluation of the compound of Formula I in combination with a PD-1 inhibitor. Based on the nonclinical results, the addition of pembrolizumab to the compound of Formula I may enhance the re-sensitization in patients that have developed secondary resistance to treatment with PD-1 inhibitors by triggering a release in the break of the Type I interferon signaling suppression. The nonclinical data generated with this combination forms the foundation of a new treatment regimen and an opportunity for patients with advanced metastatic SCCL that have developed secondary resistance to access a new treatment option.

In terms of safety, the compound of Formula I has been administered to 50 patients in the on-going first-in-human Phase 1 trial and has demonstrated to have a favorable safety profile. Overall, the safety profile of the compound of Formula I is considered manageable at the RP2D for combination with pembrolizumab.

The toxicity profile of pembrolizumab as a monoclonal antibody including irAEs is well known and it differs from the observed compound of Formula I emerging profile. However, given the fact that the compound of Formula I also acts as an immune modulator, it is anticipated that any potential for overlapping toxicities with the combination, should they occur, can be monitored in the clinic with routine clinical observations, laboratory tests, and clinical pathological assessments if needed.

Given available data with pembrolizumab, and the knowledge that it is cleared by catabolism, no DDIs are expected to occur with the combination. However, an effect of combination treatment on the PK and an exposure response of the compound of Formula I cannot be ruled out and, therefore sparse sampling will be performed.

### Study Drug Packaging and Labeling

Each bottle of the compound of Formula I contains thirty (30) 100 mg tablets for oral administration.

Each vial of pembrolizumab injection contains 100 mg of pembrolizumab in 4 mL of solution (25 mg/mL). Each 1 mL of solution contains 25 mg of pembrolizumab and is formulated in: L-histidine (1.55 mg), polysorbate 80 (0.2 mg), sucrose (70 mg), and Water for Injection, USP.

### Study Drug Supply, Receipt, and Storage

Upon receipt by qualified personnel, the compound of Formula I is to be stored at room temperature (15-30° C., 59-86° F.).

Upon receipt by qualified site personnel, pembrolizumab should be stored under refrigeration at 2° C. to 8° C. (36° F. to 46° F.) in its original carton to protect from light. Pembrolizumab should not be frozen or shaken.

### Study Drug Preparation and Administration

On days when the compound of Formula I and pembrolizumab treatment coincides, the compound of Formula I will be taken before pembrolizumab infusion.

The compound of Formula I is supplied as 100 mg tablets for oral administration and does not require preparation. The compound of Formula I will be administered orally BID. Each dose of the compound of Formula I is to be taken at the same time each day under fasted conditions (i.e., at least 2 hours after last food consumption and at least 1 hour before next food consumption), with each dose separated by 12 hours (±4 hours). If the patient misses this ±4 hour window, then the dose must be omitted and documented as a missed dose. The compound of Formula I should be swallowed whole and should not be cut, crushed, or chewed. If vomiting occurs during the course of treatment, no re-dosing of the patient is allowed before the next scheduled dose.

Pembrolizumab will be prepared and administered in the specific manner described in the manufacturer's product label. Patients will receive 200 mg pembrolizumab by IV infusion Q3W. Pembrolizumab should be administered IV over a period of 30 minutes. Investigators should make every effort to target infusion-timing to be as close to 30 minutes as possible. However, given the variability of infusion pumps from site to site, a window of −5 minutes and +10 minutes is permitted (i.e., infusion time is 30 minutes: −5 min/+10 min). Therefore, recording of infusion start and stop times is required for adherence to the protocol-specified infusion duration. Pembrolizumab may be administered up to 3 days after Day 1 of each treatment Cycle due to administrative reasons only.

### Selection of Dose for Each Patient

The compound of Formula I was well tolerated in the first-in-human, Phase 1 escalation portion of the compound of Formula I trial and data from that support the initial 200 mg BID dose in the Phase 1b Safety Run-in. The dose may be de-escalated due to observed toxicity during the study as shown in Table 6.

TABLE 6
Dose Reduction Schedule

| Compound of | | |
|---|---|---|
| Formula I Dose | | Route and |
| Level | Dose | Frequency |
| 0 (Starting Dose) | 200 mg | PO BID |
| −1 | 100 mg | PO BID |
| −2 | 100 mg | PO QD |

Abbreviations: BID = twice daily; PO = per os (orally); QD = daily

In general, if Grade 1 events occur, continue the compound of Formula I but monitor the patient more closely. For Grade 2 or higher, the compound of Formula I may be held or discontinued. Patients will remain on-treatment with single agent compound of Formula I if there are irAEs due to the administration of pembrolizumab.

An integrated body of evidence supported the approval of the 200 mg IV Q3W regimen for pembrolizumab in different tumor types. The exposure-response relationship for efficacy and safety was maintained and the regimen was approved and is adopted for wide use as per the manufacturer's labeling.

The use of a fixed dose of pembrolizumab simplifies the dosing regimen and reduces the potential for dosing errors when used in combination with the compound of Formula I.

Table 7 displays the monitoring and dose modification guidelines for irAEs due to the compound of Formula I or pembrolizumab.

TABLE 7
Monitoring and Dose Modification Guidelines for Immune-Related Adverse
Events Due to the compound of Formula I and Pembrolizumab

| Adverse | | |
|---|---|---|
| Reaction | Severity | Treatment Modification |
| Colitis | Grade 2 or 3 diarrhea or colitis | Withhold* |
| | Grade 4 diarrhea or colitis | Permanently discontinue |
| Pneumonitis | Grade 1 pneumonitis | Continue but monitor closely |
| | Grade 2 pneumonitis | Withhold* |
| | Grade 3 or 4 pneumonitis | Permanently discontinue |
| Hepatitis | Grade 1: AST, ALT, total | Continue; monitor 1-2 times |
| | bilirubin >ULN − 1.5× ULN | weekly |
| | Grade 2: AST or ALT >3.0 | Withhold* Increase frequency of |
| | to ≤5× ULN and/or total | monitoring to every 3 days. |
| | bilirubin >1.5 to ≤3× ULN | Initiate steroids if increasing |
| | | or symptomatic. OR discontinue |
| | Grade 3 or 4: AST or ALT >5× | Permanently discontinue |
| | ULN and/or total bilirubin >3× | |
| | ULN | |
| Hypophysitis | Grade 2 or 3 hypophysitis | Withhold* |
| | Grade 4 hypophysitis | Permanently discontinue |
| Adrenal | Grade 2 adrenal insufficiency | Withhold* |
| Insufficiency | Grade 3 or 4 adrenal | Permanently discontinue |
| | insufficiency | |
| Type I | Grade 3 hyperglycemia | Withhold* |
| Diabetes | Grade 4 hyperglycemia | Permanently discontinue |
| Mellitus | | |
| Nephritis/ | Serum creatinine >1.5× | Withhold* |
| Renal | up to 6× ULN | |
| Dysfunction | Serum creatinine >6× ULN | Permanently discontinue |
| Skin | Grade 3 rash or suspected | Withhold* |
| | Stevens-Johnson Syndrome | |
| | (SJS) or toxic epidermal | |
| | necrolysis (TEN) | |
| | Grade 4 rash or confirmed SJS | Permanently discontinue |
| | or TEN | |
| Encephalitis | New onset moderate or severe | Withhold* |
| | neurologic signs or symptoms | |
| | Immune-mediated encephalitis | Permanently discontinue |

*In patients with complete or partial resolution (Grades 0 to 1) after corticosteroid taper, resume the compound of Formula I at a reduced dose. Permanently discontinue the compound of Formula I if no complete or partial resolution within 21 daysof initiating steroids or inability to reduce prednisone to 10 mg per day or less (or equivalent) within 21 days of initiating steroids.
Abbreviations: ALT = alanine transaminase; AST = aspartate transaminase; SJS = Stevens-Johnson Syndrome; TEN = toxic epidermal necrolysis; ULN = upper limit of normal

Pembrolizumab dose reductions are not permitted. Pembrolizumab treatment may be interrupted or discontinued due to toxicity or interrupted in the case of medical/surgical events or logistical reasons not related to study therapy (e.g., elective surgery, unrelated medical events, patient vacation, and/or holidays). Patients should be placed back on study therapy within 3 weeks of the scheduled interruption, unless otherwise discussed with the Sponsor. The reason for interruption should be documented in the patient's study record.

If a dose of pembrolizumab is withheld for combination treatment-related toxicity, the patient may resume dosing at the following scheduled dosing appointment or when toxicity has improved as described in Table 8.

Pembrolizumab will be withheld for combination treatment-related Grade 4 hematologic toxicities, non-hematologic toxicities Grade 3 including laboratory abnormalities, and severe or life-threatening AEs (Table 8).

TABLE 8
Pembrolizumab Dose Modification Guidelines for
Combination Treatment-Related Adverse Events

| | | | Timing | | Discontinue |
|---|---|---|---|---|---|
| | | | for | Schedule for | Patient (after |
| | | Hold | restarting | restarting | consultation with |
| Toxicity | Grade | Treatment | treatment | treatment | Sponsor) |
| Hematological | 1, 2, 3 | No | N/A | N/A | N/A |
| Toxicity | 4 | Yes | Toxicity | | Toxicity does not |
| | | | resolved to | | resolve within 12 |
| | | | Grade 0 or | | weeks of last |
| | | | 1 | | infusion |
| Non- | 1 | No | N/A | N/A | N/A |
| hematological | 2 | Consider | Toxicity | Clinical AE | Toxicity does not |
| Toxicity | | withholding | resolves to | resolves within 4 | resolve within 12 |
| | | for | Grade 0 or | weeks: restart as | weeks of last |
| | | persistent | 1 | per the schedule | infusion |
| | | symptoms | | Clinical AE does | |
| | | | | not resolve within | |
| | | | | 4 weeks: | |
| | | | | Consider | |
| | | | | delaying the start | |
| | | | | ofthe next cycle | |
| | 3, 4 | Yes | Toxicity | Delay the start of | Toxicity does not |
| | | | resolved to | the next cycle by | resolve within 12 |
| | | | Grade 0 or | 3 weeks (i.e., 1 | weeks of last |
| | | | 1 | treatment cycle | infusion |
| | | | | delay) | |

Permanent discontinuation should be considered for any severe or life-threatening event
Abbreviations: N/A = not applicable

The dosage of pembrolizumab can be further modified as set forth in the FDA prescribing information for Keytruda (pembrolizumab), Reference ID: 4308423. (https://www.accessdata.fda.gov/drugsatfda_docs/label/2018/125514s046lbl.pdf).

If toxicity does not resolve to Grade 0-1 within 12 weeks after last infusion, pembrolizumab should be discontinued after consultation with the Sponsor. Patients who require corticosteroids to manage drug-related adverse events must be at an equivalent dose of ≤10 mg per day of prednisone to resume dosing with pembrolizumab. Furthermore, an inability to reduce the corticosteroid dose for managing a drug-related adverse event to the equivalent of 10 mg prednisone per day within 12 weeks of last pembrolizumab dose should prompt discussion between the investigator and Sponsor regarding the patient's ability to continue on treatment in the trial. With Investigator and Sponsor agreement, patients with a laboratory AE still at Grade 2 after 12 weeks may continue treatment in the trial only if asymptomatic and controlled. If a patient is prescribed supportive medication that is not a glucocorticoid to manage Grade 2 hypothyroidism or Grade 2 hyperthyroidism, as long as the patient is asymptomatic, and clinically stable on the supportive medication, the patient may continue receiving study medication.

In patients who continue on pembrolizumab having experienced a Grade 3, Grade 4, or persistent (>4 weeks) Grade 2 drug-related AE, once the patient has recovered from the AE to Grade 0-1, the start of the next cycle may be delayed by 3 weeks (i.e., 1 treatment cycle delay). However, in patients who experience Grade 3 or 4 pneumonitis, or recurrent persistent (>4 weeks) Grade 2 drug-related pneumonitis after rechallenge from a prior episode of persistent (>4 weeks) Grade 2 drug-related pneumonitis, pembrolizumab must be permanently discontinued.

For patients who experience a recurrence of the same AE(s) at the same grade or greater with rechallenge of pembrolizumab, a consultation between the Sponsor and investigator should occur to determine whether the patient should continue in the trial. However, for a patient who experiences a recurrence of the same SAE at the same grade or greater with rechallenge of pembrolizumab, the patient must discontinue study medication.

Adverse events (both non-serious and serious) associated with pembrolizumab exposure may represent an immunologic etiology. These adverse events may occur shortly after the first dose or several months after the last dose of treatment.

### Laboratory Assessments for Pharmacokinetics and Disease Evaluation

Serial blood samples will be drawn for assessment of the compound of Formula I concentration in plasma. Samples may be collected at any point during the specified windows. The time of day of the collection will be recorded.

Blood samples for PK analysis will be obtained at the following timepoints (Cycle (C); day (D)):
Phase 1b Safety Run-in:
   C1D1
      After dosing with the compound of Formula I
      1-2 hours post-dose (±5 minutes)
      4 hours (−30 minutes/+2 hours) post-dose
   C1D8
      Before dosing with the compound of Formula I
      After dosing with the compound of Formula I
         1-2 hours post-dose (±5 minutes)
   C1D15
      Before dosing with the compound of Formula I
         After dosing with the compound of Formula I
         1-2 hours post-dose (±5 minutes)
   C2D1
      Before dosing with the compound of Formula I
Phase 2:
   C1D1
      After dosing with the compound of Formula I
         1-2 hours post-dose (±5 minutes)
         4 hours (−30 minutes/+2 hours) post-dose
   C1D15
      Before dosing with the compound of Formula I
         After dosing with the compound of Formula I
         1-2 hours post-dose (±5 minutes)
   C2D1
      Before dosing with the compound of Formula I

Blood samples and fresh tumor samples are to be collected for exploratory biomarker analyses. Such blood samples and tumor tissue collected pre-dose will be analyzed to assess genetic or biochemical parameters that may correlate with response to the compound of Formula I (e.g., circulating tumor deoxyribonucleic acid [DNA], PARP7 mRNA or protein levels, and potential ribonucleic acid [RNA] expression profiling or other analysis if sufficient tissue samples are available).

Tumor-specific genotyping and/or expression level testing for relevant cancer genes also may be performed for patients for whom such data are not already available.

Patient consent for tumor biopsy collection during Screening is mandatory. Procedures to collect biopsies for which the risk of mortality or major morbidity, in the patient's clinical setting and at the institution completing the procedure, is 2% or higher must not be utilized. Archival biopsy samples collected within 12 weeks prior to Screening are acceptable as the baseline biopsy. In the event that collection of a fresh biopsy is not medically feasible or in cases where the fresh biopsy is deemed not evaluable (e.g., no tumor present), submission of an archival sample collected >12 weeks prior to Screening may be requested.

An optional collection of an on treatment biopsy is highly encouraged at C2D1 (±1 week) to explore pharmacodynamic (Pd) changes in the tumor microenvironment pertaining to the Type I IFN response (e.g., ISG expression) and effects on the adaptive immune response (e.g., CD8+ T cells, Granzyme B expression).

All samples will be stored until such time that appropriate analyses are developed/identified (maximum of up to 15 years).

Peripheral blood samples for exploratory Pd analyses will be collected at the following timepoints:

### Phase 1b Safety Run-In:

C1D1
Before dosing with the compound of Formula I
After dosing with the compound of Formula I
   1-2 hours (±5 minutes) post-dose
   4 hours (±30 minutes) post-dose

C1D8
Before dosing with the compound of Formula I

C1D15
Before dosing with the compound of Formula I

C2D1
Before dosing with the compound of Formula I

### Phase 2:

C1D1
Before dosing with the compound of Formula I

C1D15
Before dosing with the compound of Formula I

C2D1
Before dosing with the compound of Formula I

### Clinical Activity Measurements

Clinical activity will be measured by radiographic assessments to determine tumor measurements and disease response at scheduled timepoints.

Radiographic assessments will be centrally collected and stored by a Sponsor-designated CRO. The Sponsor may elect to independently review the scans by central read as defined by charter.

During Screening, tumor lesions are to be categorized as measurable versus non-measurable and target versus non-target, as follows.

### Measurable Versus Non-Measurable

Measurable: lesions that could accurately be measured in at least 1 dimension as ≥10 mm by CT scan or caliper measurement by clinical examination or ≥20 mm by chest X-ray; the longest diameter is to be recorded.

Non-measurable: all other lesions, including small lesions (longest diameter<10 mm or pathological lymph nodes with ≥10 to <15 mm short axis) and truly non-measurable lesions.

### Target Versus Non-Target

Target: all measurable lesions up to a maximum of 2 lesions per organ and 5 lesions in total, representative of all involved organs, are to be identified as target lesions and measured and recorded at Screening. Target lesions are to be selected on the basis of their size (i.e., those with the longest diameter) and suitability for accurate repeated measurement. The sum of the longest diameter for all target lesions is to be calculated and recorded in the eCRF as the baseline sum longest diameter.

Non-target: all other lesions not classified as target lesions (or sites of disease) are to be identified as non-target lesions and are to be recorded in the eCRF. Measurement of non-target lesions is not required.

For patients with measurable disease, disease response assessments are to be performed within 28 days prior to C1D1 and repeated within 5 days before to 7 days after the first study drug dose in every other cycle starting from C3D1. After 1 year, disease response assessments will be conducted every 9 weeks.

Disease response in target and non-target lesions will be assessed using RECIST guidelines, version 1.1, according to the categories and criteria described in Table 9. The best overall response for each patient will be reported as the best response documented over the sequence of objective statuses recorded using the categories and criteria in Table 10. If atypical imaging responses are observed, e.g., delayed response, transient tumor enlargement followed by shrinkage, stable size, or initial appearance of new lesions followed by stability or response, the Sponsor may conduct additional unplanned irRECIST assessments.

Any patient with a CR or PR is to have repeat assessments performed 4 weeks after the initial assessment to confirm the response.

TABLE 9
Response Evaluation Criteria in Solid Tumors
(RECIST) Guidelines for Tumor Response
Disease Response Criteria for Target and Nontarget Lesions
Evaluation of Target Lesions

| | |
|---|---|
| Complete Response | Disappearance of all target lesions. |
| (CR): | |
| Partial Response | At least a 30% decrease in the sum of the LD |
| (PR): | of target lesions, taking as reference the |
| | baseline sum LD. |
| Stable Disease | Neither sufficient shrinkage to qualify |
| (SD): | for PR nor sufficient increase to qualify |
| | for PD, taking as reference the smallest |
| | sum LD since the treatment started. |
| Progressive | At least a 20% increase in the sum of the |
| Disease (PD): | LD of target lesions, taking as reference |
| | the smallest sum LD recorded since the |
| | treatment started or the appearance of |
| | one or more new lesions. |

Evaluation of Nontarget Lesions

| | |
|---|---|
| Complete | Disappearance of all nontarget lesions and |
| Response (CR): | normalization of tumor marker level. |
| Incomplete Response/ | Persistence of one or more nontarget |
| Stable Disease (SD): | lesion(s) or/and maintenance of tumor |
| | marker level above the normal limits. |
| Progressive | Appearance of one or more new lesions |
| Disease (PD): | and/or unequivocal progression of existing |
| | nontarget lesions. |

Eisenhauer E A, Therasse P, Bogaerts J, Schwartz L H, Sargent D, Ford R, et al. New response evaluation criteria in solid tumours: revised RECIST guideline (version 1.1). Eur J Cancer 2009; 45(2): 228-47. Available at: http://www.eortc.be/recist/documents/RECISTGuidelines.pdf
Abbreviations: CR = complete response; PD = progressive disease; PR = partial response; SD = stable disease; LD = longest diameter.

TABLE 10
Overall Response Criteria
Patients with Target and Nontarget Lesions

| | | New | Overall |
|---|---|---|---|
| Target Lesions | Nontarget Lesions | Lesions | Response |
| CR | CR | No | CR |
| CR | Non-CR/Non-PD | No | PR |
| CR | Not evaluated | No | PR |
| PR | Non-PD or not all | No | PR |
| | evaluated | | |
| SD | Non-PD or not all | No | SD |
| | evaluated | | |
| Not evaluated | Non-PD | No | NE |
| PD | Any | Yes or no | PD |
| Any | PD | Yes or no | PD |
| Any | Any | Yes | PD |

Patients with Nontarget Lesions Only

| Non-Target | | |
|---|---|---|
| Lesions | New Lesions | Overall Response |
| CR | No | CR |
| Non-CR/Non-PD | No | Non-CR/Non-PD |
| Not all evaluated | No | NE |
| Unequivocal PD | Yes or no | PD |
| Any | Yes | PD |

Eisenhauer E A, Therasse P, Bogaerts J, Schwartz L H, Sargent D, Ford R, et al. New response evaluation criteria in solid tumours: revised RECIST guideline (version 1.1). Eur J Cancer 2009; 45(2): 228-47. Available at: http://www.eortc.be/recist/documents/RECISTGuidelines.pdf
Abbreviations: CR = complete response; NE = inevaluable; PD = progressive disease; PR = partial response; SD = stable disease

### Safety

An Adverse Event (AE) is defined in the International Council for Harmonisation (ICH) Guideline for Good Clinical Practice (GCP) as “any untoward medical occurrence in a patient or clinical investigation subject administered a pharmaceutical product and that does not necessarily have a causal relationship with this treatment” (ICH E6:1.2).

Worsening of a pre-existing medical condition, (e.g., diabetes, migraine headaches, gout) after the first dose is to be considered an AE if there is either an increase in severity, frequency, or duration of the condition or an association with significantly worse outcomes.

Interventions for pretreatment conditions (e.g., elective cosmetic surgery) or medical procedures that were planned before study enrollment are not considered AEs.

In the case of death, only record “Fatal” for the event causing death. AEs that are ongoing at the end of the study or time of death are to be noted as “continuing.”

The Investigator is responsible for reviewing laboratory test results and determining whether an abnormal value in an individual patient represents a significant change from baseline. In general, abnormal laboratory findings without clinical significance (based on the Investigator's judgment) should not be recorded as AEs; however, laboratory value changes requiring therapy or adjustment in prior therapy are considered AEs.

For the purposes of this study, death and disease progression (i.e., PD) are not considered AEs and should not be reported as such. Death is considered an outcome of one or more primary AEs, and disease progression is considered a worsening of underlying disease and is a criterion for study drug discontinuation. PD is not to be recorded as an AE.

A suspected adverse reaction is any AE for which there is a reasonable possibility that the drug caused the AE. For the purposes of Investigational New Drug Application (IND) safety reporting, “reasonable possibility” and/or at least possibly related means there is evidence to suggest a causal relationship between the drug and the AE. A “suspected adverse reaction” implies a lesser degree of certainty about causality than “adverse reaction,” which means any AE caused by a drug.

An AE or suspected adverse reaction is considered “serious” if, in the view of either the Investigator or Sponsor, it results in any of the following outcomes:
Is fatal.
Is life-threatening (i.e., places the patient at immediate risk of death).
Requires in-patient hospitalization (overnight stay) or prolongation of existing hospitalization.
Results in persistent or significant disability/incapacity.
Results in a congenital anomaly/birth defect in the offspring of a study patient.
Is an important medical event; an important medical event is an event that may not result in death, be life-threatening, or require hospitalization but may be considered an SAE when, based upon appropriate medical judgment, it may jeopardize the patient and may require medical or surgical intervention to prevent one of the outcomes listed in the definitions for SAEs. Examples of such medical events include allergic bronchospasm requiring intensive treatment in an emergency room or at home, blood dyscrasias or convulsions that do not result in inpatient hospitalization, or the development of drug dependency or drug abuse.

A hospitalization meeting the regulatory definition for “serious” is any inpatient hospital admission that includes a minimum of an overnight (≥24 hours) stay in a health care facility. Any AE that does not meet one of the definitions of serious (e.g., emergency room visit, out-patient surgery, or requires urgent investigation) may be considered by the Investigator to meet the “important medical event” criterion for classification as an SAE.

An AE or suspected adverse reaction is considered “unexpected” if it is not listed in the IB or is not listed at the specificity or intensity that has been previously observed; or, if an IB is not required or available, is not consistent with the risk information described in the General Investigational Plan or elsewhere in the current application, as amended.

An AE or suspected adverse reaction is considered “unexpected” if it is not listed in the IB or is not listed at the specificity or intensity that has been previously observed; or, if an IB is not required or available, is not consistent with the risk information described in the General Investigational Plan or elsewhere in the current application, as amended.

A serious and unexpected suspected adverse reaction (SUSAR) is any event that meets all 3 of the following definitions:

1. suspected adverse reaction;

2. serious; and

3. unexpected.

The relationship between the study drug and the AE will be described using one of the following categories as presented in Table 11.

TABLE 11
Criteria for Determination of Adverse Event Relationship
to Study Drug (compound of Formula I and/or Pembrolizumab
[Individually or in Combination])

| Relationship | Definition |
|---|---|
| Related | The study drug is more likely the cause of the AE |
| | than other factors |
| Possibly | There is a reasonable possibility that the study |
| Related | drug is the cause of the AE, including that the |
| | study drug and other factor(s) are equally likely |
| | as causes of the AE |
| Unlikely | Another factor is considered more likely the cause |
| Related | of the AE than the study drug |
| Not related | Another factor is considered to be the cause of the AE |

Abbreviations: AE = adverse event

The intensity of each AE is to be assessed by the Investigator using the NCI CTCAE, version 5.0 (National Cancer Institute 2017).

AEs not listed on the NCI CTCAE are to be rated by the Investigator according to the categories in Table 12.

TABLE 12
Criteria for Determination of Adverse Event Intensity

| Intensity | Definition |
|---|---|
| Mild | Asymptomatic or mild symptoms: clinical or |
| (Grade 1): | diagnostic observations only; intervention |
| | not indicated. |
| Moderate | Minimal, local, or non-invasive intervention |
| (Grade 2) | indicated; limiting age-appropriate instrumental |
| | activities of daily living. |
| Severe | Severe or medically significant but not immediately |
| (Grade 3): | life-threatening: hospitalization or prolongation of |
| | hospitalization indicated; disabling; limiting self-care |
| | activities of daily living; incapacitating with inability |
| | to work or perform normal daily activity. |
| Life-threatening | Consequences: urgent intervention indicated. |
| (Grade 4): | |
| Fatal (Grade 5) | AE resulted in death |

Abbreviations: AE = adverse event

The outcome of each AE will be described using the categories in Table 13.

TABLE 13
Criteria for Determination of Adverse Event Outcome

| Outcome | Definition |
|---|---|
| Resolved without | The event resolved and patient returned to baseline |
| sequelae: | |
| Resolved with | The event resolved but the patient is left with |
| sequelae: | residual problems (e.g., functional deficits, pain) |
| Resolving | At the last observation, the event was improving |
| Not Resolved | At the last observation, the event was unchanged |
| Death (Fatal) | To be used for the one AE which, in the judgment |
| | of the Investigator, was the primary cause of death |
| Unknown | There were no observations after the onset |
| | (initial observation or report) of the event |

Abbreviations: AE = adverse event; e.g. = for example

For each AE, the Investigator will indicate the action taken regarding the administration of study drug (the compound of Formula I or pembrolizumab) per the categories in Table 14.

TABLE 14
Study Drug Action Taken as a Result of Adverse Events

| Action | Definition |
|---|---|
| Discontinued | Study drug was stopped permanently due to the AE |
| (withdrawn) | |
| Dosing | Study drug regimen was modified by being temporarily |
| Interrupted | halted, i.e., one or more doses were not administered, |
| | but drug was not stopped permanently |
| Dose | Study drug regimen was modified by subtraction, i.e., |
| Decreased | by decreasing the frequency, strength or amount |
| None | No change in the administration of study drug |

Abbreviations: AE = adverse event; i.e. = in other words

Clinically significant abnormal laboratory findings or other abnormal diagnostic assessments (e.g., ECGs, vital signs, etc.) that are detected in participants who receive the compound of Formula I and/or pembrolizumab or that significantly worsen relative to Baseline in participants who receive the compound of Formula I and/or pembrolizumab will be reported as AEs. Clinical significance is based on the Investigator's judgment but will typically include findings that require active medical management or are associated with clinical signs.

Pregnancies (in female study patients or the partner of a male study patient) occurring while the patient is receiving study drug or within 30 days after the patient's last study drug dose will not be considered serious, but are to be reported using the same procedures as for SAEs described above.

AEs will be collected and recorded in this study from the time of the first dose of study drug administration through 30 days after the last dose of study drug. Collection and recording of irAEs will continue through 90 days after the last dose of study drug. Every effort will be made to collect and record events through direct contact.

Various modifications of the invention, in addition to those described herein, will be apparent to those skilled in the art from the foregoing description. Such modifications are also intended to fall within the scope of the appended claims. Each reference cited in the present disclosure, including all patent, patent applications, and publications, is incorporated herein by reference in its entirety.

What is claimed is:

1. A method for treating cancer in a subject, wherein the method comprises administering to the subject the compound 5-[[(2S)-1-(3-oxo-3-[4-[5-(trifluoromethyl)pyrimidin-2-yl]piperazin-1-yl]propoxy)propan-2-yl]amino]-4-(trifluoromethyl)-2,3-dihydropyridazin-3-one, or a pharmaceutically acceptable salt thereof, at a total daily dosage of about 50 mg to about 1000 mg, measured as the free base.

2. The method of claim 1, wherein the compound, or pharmaceutically acceptable salt thereof, is administered at a total daily dosage of about 50 mg, measured as the free base.

3. The method of claim 1, wherein the compound, or pharmaceutically acceptable salt thereof, is administered at a total daily dosage of about 100 mg, measured as the free base.

4. The method of claim 1, wherein the compound, or pharmaceutically acceptable salt thereof, is administered at a total daily dosage of about 200 mg, measured as the free base.

5. The method of claim 1, wherein the compound, or pharmaceutically acceptable salt thereof, is administered at a total daily dosage of about 400 mg, measured as the free base.

6. The method of claim 1, wherein the compound, or pharmaceutically acceptable salt thereof, is administered at a total daily dosage of about 600 mg, measured as the free base.

7. The method of claim 1, wherein the compound, or pharmaceutically acceptable salt thereof, is administered at a total daily dosage of about 800 mg, measured as the free base.

8. The method of claim 1, wherein the compound, or pharmaceutically acceptable salt thereof, is administered once daily.

9. The method of claim 1, wherein the compound, or pharmaceutically acceptable salt thereof, is administered twice daily.

10. The method of claim 1, wherein the compound, or a pharmaceutically acceptable salt thereof, is administered according to a continuous dosing schedule.

11. The method of claim 10, wherein the continuous dosing schedule comprises administering the compound, or a pharmaceutically acceptable salt thereof, to the subject for two or more consecutive days.

12. The method of claim 10, wherein the continuous dosing schedule comprises administering the compound, or a pharmaceutically acceptable salt thereof, to the subject for five or more consecutive days.

13. The method of claim 10, wherein the continuous dosing schedule comprises administering the compound, or a pharmaceutically acceptable salt thereof, to the subject for ten or more consecutive days.

14. The method of claim 10, wherein the continuous dosing schedule comprises administering the compound, or a pharmaceutically acceptable salt thereof, to the subject for fifteen or more consecutive days.

15. The method of claim 10, wherein the continuous dosing schedule comprises administering the compound, or a pharmaceutically acceptable salt thereof, to the subject for twenty or more consecutive days.

16. The method of claim 10, wherein the continuous dosing schedule comprises administering the compound, or a pharmaceutically acceptable salt thereof, to the subject for twenty-one or more consecutive days.

17. The method of claim 1, wherein the compound, or a pharmaceutically acceptable salt thereof, is administered according to an intermittent dosing schedule.

18. The method of claim 17, wherein the intermittent dosing schedule comprises administering the compound, or a pharmaceutically acceptable salt thereof, to the subject for a treatment period of two or more consecutive days followed by a period of at least one day of no treatment with the compound, or a pharmaceutically acceptable salt thereof.

19. The method of claim 18, wherein the treatment period is at least seven consecutive days.

20. The method of claim 18, wherein the treatment period is 7 to 21 consecutive days.

21. The method of claim 18, wherein the treatment period is 14 consecutive days.

22. The method of claim 18, wherein the period of no treatment is at least 2 consecutive days.

23. The method of claim 18, wherein the period of no treatment is at least 4 consecutive days.

24. The method of claim 18, wherein the period of no treatment is 4 to 10 consecutive days.

25. The method of claim 18, wherein the period of no treatment is 7 consecutive days.

26. The method of claim 18, wherein the intermittent dosing schedule comprises at least two treatment periods, where all treatment periods are separated by a period of no treatment.

27. The method of claim 9, wherein the patient is administered twice daily a dosage of 50 mg of the compound, or a pharmaceutically acceptable salt thereof, measured as the free base.

28. The method of claim 9, wherein the patient is administered twice daily a dosage of 100 mg of the compound, or a pharmaceutically acceptable salt thereof, measured as the free base.

29. The method of claim 9, wherein the patient is administered twice daily a dosage of 200 mg of the compound, or a pharmaceutically acceptable salt thereof, measured as the free base.

30. The method of claim 9, wherein the patient is administered twice daily a dosage of 300 mg of the compound, or a pharmaceutically acceptable salt thereof, measured as the free base.

31. The method of claim 9, wherein the patient is administered twice daily a dosage of 400 mg of the compound, or a pharmaceutically acceptable salt thereof, measured as the free base.

32. The method of claim 9, wherein the patient is administered twice daily a dosage of 500 mg of the compound, or a pharmaceutically acceptable salt thereof, measured as the free base.

33. The method of claim 1, wherein the cancer is a breast cancer, a cancer of the central nervous system, an endometrium cancer, a kidney cancer, a large intestine cancer, a lung cancer, an oesophagus cancer, an ovarian cancer, a pancreatic cancer, a prostate cancer, a stomach cancer, a head and neck cancer, an urinary tract cancer, a colon cancer, or a cancer in which PARP7 expression is amplified.

34. The method of claim 1, wherein the compound, or a pharmaceutically acceptable salt thereof, is administered orally.

35. The method of claim 34 wherein the compound, or a pharmaceutically acceptable salt thereof, is administered as an oral unit dosage form.

36. The method of claim 35, wherein the oral unit dosage form is in the form of a capsule or tablet.

37. The method of claim 36, wherein the compound, or a pharmaceutically acceptable salt thereof, has a particle diameter [d90] of less than about 30 μm.

38. The method of claim 36, wherein the compound, or a pharmaceutically acceptable salt thereof, has a particle diameter [d90] of about 1 to about 20 μm.

39. The method of claim 36, wherein the compound, or a pharmaceutically acceptable salt thereof, has a particle diameter [d90] of about 5 to about 15 μm.

40. The method of claim 36, wherein the compound, or a pharmaceutically acceptable salt thereof, has a particle diameter [d90] of about 7 to about 12 μm.

41. The method of claim 36, wherein the micronized compound, or a pharmaceutically acceptable salt thereof, has a particle diameter [d90] of about 8, 9, 10, or 11 μm.

42. A unit dosage form comprising the compound 5-[[(2S)-1-(3-oxo-3-[4-[5-(trifluoromethyl)pyrimidin-2-yl]piperazin-1-yl]propoxy)propan-2-yl]amino]-4-(trifluoromethyl)-2,3-dihydropyridazin-3-one,
or a pharmaceutically acceptable salt thereof, in an amount of about 25 mg to about 500 mg, measured as the free base.

43. The unit dosage form of claim 42, which is suitable for oral administration.

44. The unit dosage form of claim 42, comprising the compound, or a pharmaceutically acceptable salt thereof, in an amount of about 25 mg, measured as the free base.

45. The unit dosage form of claim 42, comprising the compound, or a pharmaceutically acceptable salt thereof, in an amount of about 50 mg, measured as the free base.

46. The unit dosage form of claim 42, comprising the compound, or a pharmaceutically acceptable salt thereof, in an amount of about 75 mg, measured as the free base.

47. The unit dosage form of claim 42, comprising the compound, or a pharmaceutically acceptable salt thereof, in an amount of about 100 mg, measured as the free base.

48. The unit dosage form of claim 42, comprising the compound, or a pharmaceutically acceptable salt thereof, in an amount of about 150 mg, measured as the free base.

49. The unit dosage form of claim 42, comprising the compound, or a pharmaceutically acceptable salt thereof, in an amount of about 200 mg, measured as the free base.

50. The unit dosage form of claim 42, comprising the compound, or a pharmaceutically acceptable salt thereof, in an amount of about 250 mg, measured as the free base.

51. The unit dosage form of claim 42, comprising the compound, or a pharmaceutically acceptable salt thereof, in an amount of about 300 mg, measured as the free base.

52. The unit dosage form of claim 42, wherein the unit dosage form is in the form of a capsule or a tablet.

53. The unit dosage form of claim 52 wherein the particle size diameter [d90] of the compound, or pharmaceutically acceptable salt thereof, is less than about 30 μm.

54. The unit dosage form of claim 52 wherein the particle size diameter [d90] of the compound, or pharmaceutically acceptable salt thereof, is about 1 to about 20 μm.

55. The unit dosage form of claim 52 wherein the particle size diameter [d90] of the compound, or pharmaceutically acceptable salt thereof, is about 5 to about 15 μm.

56. The unit dosage form of claim 52 wherein the particle size diameter [d90] of the compound, or pharmaceutically acceptable salt thereof, is about 7 to about 12 μm.

57. The unit dosage form of claim 52 wherein the particle size diameter [d90] of the compound, or a pharmaceutically acceptable salt thereof, is about 8, 9, 10, or 11 μm.

58. A method for treating cancer in a subject, wherein the method comprises administering to the subject a pharmaceutical composition that comprises the compound 5-[[(2S)-1-(3-oxo-3-[4-[5-(trifluoromethyl)pyrimidin-2-yl]piperazin-1-yl]propoxy)propan-2-yl]amino]-4-(trifluoromethyl)-2,3-dihydropyridazin-3-one, or a pharmaceutically acceptable salt thereof, in an amount sufficient to provide at steady-state:
(a) a mean Cₘₐₓ of about 140 ng/mL to about 1600 ng/mL;
(b) a mean Cₘᵢₙ of at least about 50 ng/mL to 700 ng/mL;
(c) a mean Tₘₐₓ of about 1 hour to 5 hours; or
(d) a mean AUC₀₋₁₂ of about 800 hr*ng/mL to 10500 hr*ng/mL.

59. The method of claim 58, wherein the compound, or a pharmaceutically acceptable salt thereof, is administered in an amount sufficient to provide at steady-state, a mean Cₘₐₓ of about 200 ng/mL to about 800 ng/mL.

60. The method of claim 58, wherein the compound, or a pharmaceutically acceptable salt thereof, is administered in an amount sufficient to provide at steady-state, a mean Cₘₐₓ of about 300 ng/mL to about 800 ng/mL.

61. The method of claim 58, wherein the compound, or a pharmaceutically acceptable salt thereof, is administered in an amount sufficient to provide at steady-state, a mean Cₘₐₓ of about 400 ng/mL to about 700 ng/mL.

62. The method of claim 58, wherein the compound, or a pharmaceutically acceptable salt thereof, is administered in an amount sufficient to provide at steady-state, a mean Cₘₐₓ of about 500 ng/mL to about 700 ng/mL.

63. The method of claim 58, wherein the compound, or a pharmaceutically acceptable salt thereof, is administered in an amount sufficient to provide at steady-state, a mean Cₘᵢₙ of about 200 ng/mL to about 500 ng/mL.

64. The method of claim 58, wherein the compound, or a pharmaceutically acceptable salt thereof, is administered in an amount sufficient to provide at steady-state, a mean Cₘᵢₙ of about 300 ng/mL to about 500 ng/mL.

65. The method of claim 58, wherein the compound, or a pharmaceutically acceptable salt thereof, is administered in an amount sufficient to provide at steady-state, a mean Cₘᵢₙ of about 300 ng/mL to about 400 ng/mL.

66. The method of claim 58, wherein the compound, or a pharmaceutically acceptable salt thereof, is administered in an amount sufficient to provide at steady-state, a mean Tₘₐₓ of about 2 hours to 4 hours.

67. The method of claim 58, wherein the compound, or a pharmaceutically acceptable salt thereof, is administered in an amount sufficient to provide at steady-state, a mean Tₘₐₓ of about 2 hours to 3 hours.

68. The method of claim 58, wherein the compound, or a pharmaceutically acceptable salt thereof, is administered in an amount sufficient to provide at steady-state, a mean AUC_{0-12 h} of about 1000 hr*ng/mL to 5000 hr*ng/mL.

69. The method of claim 58, wherein the compound, or a pharmaceutically acceptable salt thereof, is administered in an amount sufficient to provide at steady-state, a mean AUC_{0-12 h} of about 1400 hr*ng/mL to 4500 hr*ng/mL.

70. The method of claim 58, wherein the compound, or a pharmaceutically acceptable salt thereof, is administered in an amount sufficient to provide at steady-state, a mean AUC_{0-12 h} of about 1200 hr*ng/mL to 4000 hr*ng/mL.

71. A solid preparation of the compound 5-[[(2S)-1-(3-oxo-3-[4-[5-(trifluoromethyl)pyrimidin-2-yl]piperazin-1-yl]propoxy)propan-2-yl]amino]-4-(trifluoromethyl)-2,3-dihydropyridazin-3-one, or a pharmaceutically acceptable salt thereof, wherein the particle size diameter [d90] of the compound, or a pharmaceutically acceptable salt thereof, is less than about 30 μm.

72. The solid preparation of claim 71 wherein the particle size diameter [d90] of the compound, or a pharmaceutically acceptable salt thereof, is about 1 to about 20 μm.

73. The solid preparation of claim 71 wherein the particle size diameter [d90] of the compound, or a pharmaceutically acceptable salt thereof, is about 5 to about 15 μm.

74. The solid preparation of claim 71 wherein the particle size diameter [d90] of the compound, or a pharmaceutically acceptable salt thereof, is about 7 to about 12 μm.

75. The solid preparation of claim 71 wherein the particle size diameter [d90] of the compound, or a pharmaceutically acceptable salt thereof, is about 8, 9, 10, or 11 μm.

76. A solid pharmaceutical formulation comprising the compound 5-[[(2S)-1-(3-oxo-3-[4-[5-(trifluoromethyl)pyrimidin-2-yl]piperazin-1-yl]propoxy)propan-2-yl]amino]-4-(trifluoromethyl)-2,3-dihydropyridazin-3-one, or a pharmaceutically acceptable salt thereof, in an amount of about 30% to about 50% w/w, and at least one pharmaceutically acceptable excipient.

77. The pharmaceutical formulation of claim 76 wherein the compound, or a pharmaceutically acceptable salt thereof, is present in an amount of about 40% w/w.

78. The pharmaceutical formulation of claim 77 wherein the particle size diameter [d90] of the compound, or pharmaceutically acceptable salt thereof, is less than about 30 μm.

79. The pharmaceutical formulation of claim 77 wherein the particle size diameter [d90] of the compound, or pharmaceutically acceptable salt thereof, is about 1 to about 20 μm.

80. The pharmaceutical formulation of claim 77 wherein the particle size diameter [d90] of the compound, or pharmaceutically acceptable salt thereof, is about 5 to about 15 μm.

81. The pharmaceutical formulation of claim 77 wherein the particle size diameter [d90] of the compound, or pharmaceutically acceptable salt thereof, is about 7 to about 12 μm.

82. The pharmaceutical formulation of claim 77 wherein the particle size diameter [d90] of the compound, or pharmaceutically acceptable salt thereof, is about 8, 9, 10, or 11 μm.

83. A method for treating cancer in a subject, wherein the method comprises administering to the subject:
(i) the compound 5-[[(2S)-1-(3-oxo-3-[4-[5-(trifluoromethyl)pyrimidin-2-yl]piperazin-1-yl]propoxy)propan-2-yl]amino]-4-(trifluoromethyl)-2,3-dihydropyridazin-3-one,
or a pharmaceutically acceptable salt thereof; and
(ii) an antibody that binds to human PD-1.

84. The method of claim 83, wherein the compound, or a pharmaceutically acceptable salt thereof, and the antibody are administered simultaneously.

85. The method of claim 83, wherein the compound, or a pharmaceutically acceptable salt thereof, and the antibody are administered sequentially.

86. The method of claim 83, wherein the compound, or a pharmaceutically acceptable salt thereof, is administered orally.

87. The method of claim 83, wherein the antibody is administered via intravenous administration.

88. The method of claim 83, wherein the compound, or a pharmaceutically acceptable salt thereof, is administered twice daily.

89. The method of claim 83, wherein the compound, or a pharmaceutically acceptable salt thereof, is administered once daily.

90. The method of claim 83, wherein the compound, or a pharmaceutically acceptable salt thereof, is administered at a dose of about 200 mg twice daily, measured as the free base.

91. The method of claim 83, wherein the compound, or a pharmaceutically acceptable salt thereof, is administered at a dose of about 100 mg twice daily, measured as the free base.

92. The method of claim 83, wherein the compound, or a pharmaceutically acceptable salt thereof, is administered at a dose of about 100 mg once daily, measured as the free base.

93. The method of claim 83, wherein the antibody is administered once every 3 weeks.

94. The method of claim 83, wherein the antibody is administered at a dose of about 200 mg once every 3 weeks.

95. The method of claim 83, wherein the antibody is a humanized antibody.

96. The method of claim 83, wherein the antibody is pembrolizumab.

97. The method of claim 83, wherein the compound, or a pharmaceutically acceptable salt thereof, is administered according to a continuous dosing schedule.

98. The method of claim 97, wherein the continuous dosing schedule comprises administering the compound, or a pharmaceutically acceptable salt thereof, to the subject for two or more consecutive days.

99. The method of claim 97, wherein the continuous dosing schedule comprises administering the compound, or a pharmaceutically acceptable salt thereof, to the subject for five or more consecutive days.

100. The method of claim 97, wherein the continuous dosing schedule comprises administering the compound, or a pharmaceutically acceptable salt thereof, to the subject for ten or more consecutive days.

101. The method of claim 97, wherein the continuous dosing schedule comprises administering the compound, or a pharmaceutically acceptable salt thereof, to the subject for fifteen or more consecutive days.

102. The method of claim 97, wherein the continuous dosing schedule comprises administering the compound, or a pharmaceutically acceptable salt thereof, to the subject for twenty or more consecutive days.

103. The method of claim 97, wherein the continuous dosing schedule comprises administering the compound, or a pharmaceutically acceptable salt thereof, to the subject for twenty-one or more consecutive days.

104. The method of claim 83, wherein the cancer is a breast cancer, a cancer of the central nervous system, an endometrium cancer, a kidney cancer, a large intestine cancer, a lung cancer, an oesophagus cancer, an ovarian cancer, a pancreatic cancer, a prostate cancer, a stomach cancer, a head and neck cancer, an urinary tract cancer, a colon cancer, or a cancer in which PARP7 expression is amplified.

105. The method claim 104, wherein the lung cancer is squamous cell carcinoma of the lung.

106. The method of claim 83, wherein the subject has received prior treatment for cancer.

107. The method of claim 106, wherein the prior treatment is a platinum doublet chemotherapy or an immune checkpoint blockade inhibitor.

108. The method of claim 107, wherein the immune checkpoint blockade inhibitor comprises anti-PD-1/anti-PD-L1 or anti-cytotoxic T-lymphocyte-associated antigen 4 (CTLA-4) inhibitors.

109. The method of claim 83, wherein the subject has failed at least one previous treatment.
